(19) United States
(12) Patent Application Publication
(10) Pub. No.: US 2026/0284236 A1
(43) Pub. Date: Sep. 24, 2026

(54) METHODS FOR REDUCING AND/OR PREVENTING DE-CHELATION OF A CHELATED RADIOISOTOPE

(71) Applicant: Curadel Surgical Innovations, Inc., Bonita Springs, FL (US)

(72) Inventor: John V. Frangioni, Bonita Springs, FL (US)

(73) Assignee: Curadel Surgical Innovations, Inc., Bonita Springs, FL (US)

(21) Appl. No.: 19/558,370

(22) Filed: Mar. 5, 2026

Related U.S. Application Data
(60) Provisional application No. 63/767,999, filed on Mar. 6, 2025.

(51) Int. Cl. A61K 51/04

(52) U.S. Cl. CPC A61K 51/0482; A61K 51/0485; A61K 51/0495; A61K 2123/00

(57) ABSTRACT

In accordance with at least one aspect of this disclosure, a method includes reducing a probability of de-chelation of a metal or metal isotope from a chelating radiotherapeutic including a chelating radioisotope and a metal chelator during one or more of: electronic conversion of the chelating radioisotope and/or radioactive decay of the chelating radioisotope by including one or more zwitterionic group on the metal chelator.

### CROSS-REFERENCE TO RELATED APPLICATIONS

This application claims priority to and the benefit of U.S. Provisional Patent Application No. 63/767,999, filed Mar. 6, 2025, the entire content of which is incorporated by reference herein.

This application is related to U.S. patent application Ser. No. 18/640,169, filed Apr. 19, 2024, U.S. patent application Ser. No. 18/732,982, filed Jun. 4, 2024, U.S. patent application Ser. No. 18/640,620, filed Apr. 19, 2024, U.S. Provisional Patent Application No. 63/656,858, filed Jun. 6, 2024, U.S. Provisional Patent Application No. 63/648,302, filed May 16, 2024, U.S. Provisional Patent Application No. 63/724,048, filed Nov. 22, 2024, U.S. Provisional Patent Application No. 63/724,707, filed Nov. 25, 2024, U.S. Provisional Patent Application No. 63/767,817, filed Mar. 6, 2025, and U.S. application Ser. No. 19/558,087 filed Mar. 5, 2026, the entire content of each of these applications is incorporated by reference herein.

### BACKGROUND

Pb-212 is quickly emerging as one of the most important radioisotopes in cancer diagnostics and therapeutics. Its importance is derived from its simultaneous α and β emissions, with the former used to treat malignant cells and the latter permitting SPECT/CT imaging.

During the conversion of Pb-212 (oxidation state 2+) to Bi-212 (oxidation state 3+) there is significant rearrangement, i.e., electronic conversion, of the atom. For example, bismuth is thought to undergo a transient +5, and maybe even +7, oxidation state before returning to the 3+ state. During this time, bonds to the metal chelator can be broken resulting in bismuth diffusing away from the chelator. As Bi-212 itself decays, alpha particle emission occurs, with energies typically in the range of 4-10 MeV, compared to 1-10 eV for individual chelator-metal binding energies. Thus, when an alpha emission occurs, there is a very high probability that the metal will be ejected from its chelator or targeting ligand chelator complex. Both of these potential de-chelation events are undesirable when the de-chelation occurs before the chelated targeting ligand reaches the tumor because the metal can be taken up by off target tissues, such as the kidneys or bone marrow, thereby leading to high off-target radiation dose, or at least significantly increasing the risk of high off-target doses.

Mizradeh et al. (Mirzadeh et al. “The Chemical Fate of 212 Bi-DOTA Formed by/IF Decay of 212 Pb(DOTA),” Radiochimica Acta 60, 1-1 0 (1993)) studied the chemical fate of the Bi-212-DOTA complex formed by β-decay of Pb-212-DOTA using an assay that tested whether the metal chelator DOTA complexed to Pb-212 could limit the ejection of bismuth from the chelator complex. However, the assay described by Mirzadeh is extremely cumbersome, requiring micro-column purification and exact timing.

Accordingly, while conventional techniques have been considered satisfactory for their intended purpose, there is an ever present need for improved systems and methods for testing metal chelator resistance to electronic conversion as well as the ejection due to alpha particle emission. This disclosure provides a solution for this need.

### INCORPORATION BY REFERENCE

The following documents are herein incorporated by reference in their entirety:
Šimeček et al. “Efficient formation of inert Bi-213 chelates by tetraphosphorus acid analogues of DOTA: towards improved alpha-therapeutics,” EJNMMI Research (2018) 8:78, https://doi.org/10.1186/s13550-018-0431-3;
Mirzadeh et al. “The Chemical Fate of 212 Bi-DOTA Formed by/IF Decay of 212 Pb(DOTA),” Radiochimica Acta 60, 1-1 0 (1993);
Bartos et al. “Search of ligands suitable for 212 Pb/212Bi in vivo generators,” J Radioanal Nucl Chem (2013) 295:205-209, DOI 10.1007/s10967-012-2238-4;
Su et al. “Pretargeted radioimmunotherapy in tumored mice using an in vivo,” Nuclear Medicine and Biology 32 (2005) 741-747, doi:10.1016/j.nucmedbio.2005.06.009;
Ruble et al. “THE USE OF 212~-LABELED MONOCLONAL ANTIBODY IN THE TREATMENT OF MURINE ERYTHROLEUKEMIA,” Int. J. Radiation Oncology Biol. Phys., Vol. 34. No. 3. pp. 609-616, 1996; and
Bauer et al. “212 Pb-Pretargeted Theranostics for Pancreatic Cancer,” J Nucl Med 2024; 00:1-8, doi:10.2967/jnumed.123.266388.

### SUMMARY

In accordance with at least one aspect of this disclosure, a method comprises reducing the probability of de-chelation of a metal or metal isotope from a chelating radiotherapeutic, the chelating radiotherapeutic including a chelating radioisotope and a metal chelator, during one or more of: electronic conversion of the chelating radioisotope (i.e., atomic rearrangement and change in oxidation state) and/or radioactive decay of the chelating radioisotope (i.e., alpha particle emission) by including one or more zwitterionic groups on the metal chelator. In certain embodiments, one or two or three or four zwitterionic groups can be added to the metal chelator.

In accordance with at least one aspect of this disclosure, a method of improving efficacy and/or reducing an off-target dose of a chelating radiotherapeutic can include, preparing a chelating radiotherapeutic having at least a chelating radioisotope and a metal chelator, selecting the metal chelator for inclusion on the chelating radiotherapeutic based at least in part on a probability of de-chelation of a metal or metal isotope from the metal chelator during transition of the radioisotope, and further including one or more zwitterionic groups on the metal chelator. As used herein “transition” can mean an electronic conversion of the radioisotope, such as atomic rearrangement or change in oxidation state, or transition can mean radioactive decay, where excess energy is generated through alpha particle emission from the radioisotope during radioactive decay.

In certain embodiments, the metal chelator can be or include one of (or a derivative of): a DOTA-based metal chelator, a DOTAM-based metal chelator, a PCTA-based metal chelator, a DOTP-based metal chelator, an Amide-PCTA-based metal chelator, a PCTP-based metal chelator, a PCTA-based metal chelator, a Gadopiclenol-based metal chelator, a deferoxamine-based metal chelator, a NOTA-based metal chelator, a PyC3A-based metal chelator, a macropa-based metal chelator, a PYTA-based metal chelator, or a porphyrin-based metal chelator. In certain embodiments, ZWI represents a zwitterionic group. In certain embodiments, the one or more zwitterionic groups included on the metal chelator can be selected from the group consisting of: ammoniophosphates, ammoniophosphonates, ammoniophosphinates, ammoniosulfonates, ammoniosulfates, ammoniocarboxylates, ammoniosulfonamides, ammonio-sulfon-imides, guanidiniocarboxylates, pyridiniocarboxylates, pyridiniosulfonates, ammonio(alkoxy)dicyanoethenolates, ammonioboronates, sulfoniocarboxylates, phophoniosulfonates, phosphoniocarboxylates, amino carboxylic acids, amino phosphonic acids, amino phosphinic acids, amino sulfonic acids, aminoalkyl substituted sulfates, aminoalkyl substituted phosphates, betaines, N-oxide, or derivatives of sulfamic acid, and combinations thereof.

In certain embodiments, the one or more zwitterionic groups can be or include an ammonium sulfobetaine group, an N-oxide group, an ammonium sulfobetaine group bound to the chelator ring via triazole ring (i.e., “clicked” or formed by click-chemistry), or an N-oxide bound to the chelator ring through a triazole ring (i.e., “clicked). This type of click-chemistry linking of the zwitterionic groups can also be applied to any of the chelators described herein.

In certain such embodiments, the metal or metal isotope can be selected from the following: Pd, Pb, Zr, Cu, Ga, In, Y, Gd, Lu, Ac, Tb, including: Ga-66, Ga-67, Ga-68, Cu-64, Cu-67, Y-86, Co-55, Zr-89, Sr-83, Mn-52, As-72, Sc-44, Gd-153, Co-57, In-111, Ac-225, Tc-99m, Tc-94m, Re-186, Re-188, Y-90, Bi-213, Sm-153, Ho-166, Lu-177, Sc-43, Sc-44, Sc-47, Tb-149, Tb-152, Tb-155, Tb-161, Dy-166 or Pb-212.

In certain embodiments, one or more zwitterionic groups can be added to a DOTA-based metal chelator or a DOTAM-based chelator and in certain such embodiments, the metal or metal isotope can be Pd, Pb, Zr, Cu, Ga, In, Y, Gd, Lu, Ac or Tb.

In certain embodiments, one or more zwitterionic groups can be added to a PCTA-based metal chelator and in certain such embodiments, the metal or metal isotope can be Gd or Gd³⁺, Pb, Mn, Zr, Cu, Ga, In, Y, Gd, Lu, c or Tb.

In certain embodiments, one or more zwitterionic groups can be added to an amide-PCTA-based metal chelator and in certain such embodiments, the metal or metal isotope can be Gd, Gd³⁺, Pb, Mn, Zr, Cu, Ga, In, Y, Gd, Lu, c or Tb.

In certain embodiments, one or more zwitterionic groups can be added to a PCTP-based metal chelator and in certain such embodiments, the metal or metal isotope can be Pd, Pb, Mn, Zr, Cu, Ga, In, Y, Gd, Lu, Ac or Tb.

In certain embodiments, one or more zwitterionic groups can be added to a DOTP-based metal chelator and in certain such embodiments, the metal or metal isotope can be Pd, Pb, Zr, Cu, Ga, In, Y, Gd, Lu, Ac or Tb.

In certain embodiments, one or more zwitterionic groups can be added to a PyC3A-based metal chelator and in certain such embodiments, the metal or metal isotope can be Mn.

In certain embodiments, one or more zwitterionic groups can be added to a macropa-based metal chelator and in certain such embodiments, the metal or metal isotope can be Ac 3+ or Bi 3+.

In certain embodiments, one or more zwitterionic groups can be added to a NOTA-based metal chelator and in certain such embodiments, the metal or metal isotope can be Ga 3+, Cu 2+, Gd 3+, Ac 3+, or Bi 3+.

In certain embodiments, one or more zwitterionic groups can be added to a deferoxamine-based metal chelator and in certain such embodiments, the metal or metal isotope can be Zr 4+, Fe 3+, Mn 2+, or Mn 3+.

In certain embodiments, one or more zwitterionic groups can be added to a porphyrin-based metal chelator and in certain such embodiments, the metal or metal isotope can be Mn 2+, Mn 3+, Fe 2+, Fe 3+, Gd 3+, Ac 3+, or Bi 3+.

In certain embodiments, the chelating radiotherapeutic can further include one or more targeting vectors bound thereto. In certain embodiments, the one or more targeting vectors can be or include cRGD, KUE, PSMA-617, FAPI, octreotide, bombesin, or a homo- or hetero-dimer formed from their combination.

In certain embodiments, the method can further include, determining an energy delta between energy generated during transition of the radioisotope and a binding energy of the metal or metal isotope to the metal chelator and determining the probability of de-chelation of the metal or metal isotope during transition based on a magnitude of the energy delta. The method can further include, including a zwitterionic group on the metal chelator whereby inclusion of the zwitterionic group reduces the probability of de-chelation of the metal or metal isotope during transition to zero or near zero.

In certain embodiments, determining the probability of de-chelation of the metal or metal isotope during transition based on a magnitude of the energy delta can further include preparing a reference solution having a radioisotope-chelator complex, wherein the chelator complex is a reference chelator complex, measuring a radioactivity of the reference solution over a count period in counts per minute, and repeating the measuring over a plurality of time sampling intervals, and preparing a test solution having a radioisotope-chelator complex, wherein the chelator complex is a test chelator complex (in certain embodiments having one or more zwitterionic groups on the chelator complex), measuring a radioactivity of the test solution over the count period in counts per minute, and repeating the measuring over the plurality of time sampling intervals. Determining the probability of de-chelation can further include, calculating an average radioactivity for each of the reference solution and the test solution over the plurality of sampling interval and calculating a percentage of metal or metal isotope released from the test chelator complex by dividing the average radioactivity of the test solution by the average radioactivity at each energy of the reference solution for each sampling interval.

In certain embodiments, such as for determining which metal chelator is appropriate (or best suited) for use with a particular chelating radiotherapeutic, the method can further include preparing a plurality of test solutions, each test solution having a radioisotope-chelator complex, wherein the radioisotope-chelator complex is a respective test chelator complex (in certain embodiments having one or more zwitterionic groups on the chelator complex), and wherein each test chelator complex is different from one another, measuring a radioactivity of each test solution over the count period in counts per minute, and repeating the measuring over the plurality of time sampling intervals for each test solution. The method can further include, comparing the percentage of metal or metal isotope released from the chelator complex for each respective test solution to the reference solution to determine which respective chelator complex has a lowest percentage of metal or metal isotope released from the respective chelator complex.

Accordingly, in view of this determination, selecting the metal chelator for inclusion on the chelating radiotherapeutic further comprises selecting the metal chelator as a function of its respective percentage of metal or metal isotope released from the respective chelator complex, for example selecting the metal chelator that has shown the lowest probability of de-chelation for the chosen radioisotope for inclusion on the chelating radioisotope.

The inventors have found, unexpectedly, that an increase in efficacy of the radiotherapeutic and a reduction in off-target dosing can be achieved by including the selected metal chelator on the chelating radiotherapeutic and further including one or more zwitterionic group on the selected metal chelator.

The method can further include administering the chelating radiotherapeutic to a patient intravenously, and further, selecting the metal chelator having a lowest probability of de-chelation of the metal or metal isotope from the chelating radiotherapeutic wherein inclusion of the one or more zwitterionic groups on the metal chelator decreases the probability that the metal or metal isotope is de-chelated from the chelating radiotherapeutic in the patient's blood stream, before reaching a targeted tissue and thus thereby reducing an off-target radiation dose of the chelating radiotherapeutic. Said differently, selecting the metal chelator having a lowest probability of de-chelation of the metal or metal isotope from the chelating radiotherapeutic and further including the one or more zwitterionic group son the metal chelator increases the probability that the metal or metal isotope will remain bound to the chelating radiotherapeutic when the chelating radiotherapeutic reaches the target tissue, thus thereby increasing efficacy of the chelating radiotherapeutic dose.

In accordance with at least one aspect of this disclosure, an improved radiopharmaceutical for the treatment and/or diagnosis of cancer in a patient is provided. The improved radiopharmaceutical includes a chelating radiotherapeutic having (at least) a chelating radioisotope and metal chelator included on the chelating radiotherapeutic and one or more zwitterionic groups included on the metal chelator, the metal chelator selected to reduce a probability of de-chelation of a metal or metal isotope from the chelating radiotherapeutic during one or more of: electronic conversion of the chelating radioisotope and/or due to excess energy generated from alpha particle emission from radioactive decay of the chelating radioisotope thereby decreasing an off-target radiation dose and increasing targeted cell uptake maximizing an efficacy of the radiopharmaceutical.

In certain embodiments, the radiopharmaceutical can be a radiotherapeutic for the treatment of cancer in a patient.

In certain embodiments, the radiopharmaceutical can include a radiodiagnostic and the chelating radioisotope is both an alpha- and beta-emitter and is adapted for dosimetry by single-photon emission computed tomography (SPECT) imaging. In certain such embodiments, the inclusion of the one or more zwitterionic groups on the metal chelator reduces the probability of de-chelation of the metal or metal isotope from the radiodiagnostic after beta emission. Since SPECT imaging utilizes the radiation from beta particle emission, de-chelation of the metal or metal isotope after beta particle emission decreases dosimetry accuracy. Thus, including the one or more zwitterionic groups on the metal chelator of the radiodiagnostic used in SPECT will significantly reduce or prevent de-chelation of the metal or metal isotope at beta particle emission, ultimately increasing the accuracy of dosimetry.

In accordance with at least one aspect of this disclosure, an improved radiodiagnostic for the diagnosis of cancer in a patient includes a chelating radiodiagnostic having a chelating radioisotope, the chelating radioisotope being an alpha- and beta-emitter. A metal chelator is included on the chelating radioisotope and one or more zwitterionic groups are included in the metal chelator. The metal chelator is selected on a basis of a probability of de-chelation of a metal or metal isotope from the chelating radiotherapeutic during one or more of: electronic conversion of the chelating radioisotope and/or due to excess energy generated from alpha- and beta-particle emission from radioactive decay of the chelating radioisotope. In such embodiments, the inclusion of the one or more zwitterionic groups on the metal chelator reduces the probability of de-chelation of the metal or metal isotope after beta particle emission and thereby increases dosimetry accuracy during a diagnostic imaging procedure (e.g., SPECT imaging). In certain embodiments, the chelating radioisotope is Pb-212 and the metal chelator is any one or more embodiments of a metal chelator or chelator complex described herein.

In certain embodiments, the radiotherapeutic or radiodiagnostic can include one or more targeting vectors bound thereto for targeting cancer cell, wherein the one or more targeting vectors include cRGD, KUE, PSMA-617, FAPI, octreotide, bombesin, or a homo- or hetero-dimer formed from their combination. In certain embodiments, the cancer cells are melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, and pediatric brain tumor cells.

In accordance with at least one aspect of this disclosure, method of treating and/or diagnosing a patient can include administering a radiotherapeutic or radiodiagnostic having one or more embodiments of a metal chelator bound thereto, with one or more zwitterionic bound to the metal chelator, to a patient.

These and other features of the systems and methods of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, other embodiments thereof will be described in detail herein below with reference to certain figures, wherein:

FIG. 1 is a diagram showing a radioactive decay scheme of Th-228 in accordance with this disclosure;

FIG. 2 is a diagram showing the energies of the decay of Pb-212 to Pb-208;

FIG. 3 is a diagram of the decay scheme of FIG. 2, additionally showing the respective x-rays and gamma rays released;

FIG. 4 is a diagram showing the energies of the decay of Ac-225 to Bi-209;

FIG. 5 is a schematic diagram showing a desirable and undesirable condition of a chelating radiotherapeutic during transition of the radioisotope, wherein in the desirable condition, the metal or metal isotope remains attached to the chelator, and wherein in the undesirable condition, the metal or metal isotope is ejected from the chelator;

FIG. 6 is front elevation view of an exemplary embodiment of a preparation column for use in a chelator test assay in accordance with this disclosure;

FIG. 7 is a front elevation view of an exemplary embodiment of a collection column for use in the chelator test assay in accordance with this disclosure;

FIG. 8 is an isometric view of an exemplary embodiment of a pipettor for use in the chelator test assay in accordance with this disclosure;

FIG. 9 is a front elevation view of an exemplary embodiment of a column holder for use in the chelator test assay in accordance with this disclosure;

FIG. 10 is top-down oblique view of an exemplary embodiment of a column preparation rack for use in the chelator test assay in accordance with this disclosure;

FIG. 11 is a top-down oblique view of an exemplary embodiment of a reference solution rack for use in the chelator test assay in accordance with this disclosure;

FIG. 12 is a top-down oblique view of an exemplary embodiment of a column separation rack for use in the chelator test assay in accordance with this disclosure;

FIG. 13 is a simplified schematic diagram of an exemplary embodiment of the chelator test assay in accordance with this disclosure;

FIG. 14 is a logarithmic plot of F vs. time, where A₂ is the ²¹²Bi radioactivity in the EDTA chromatographic fraction of FIG. 15 and A_{2(eq)} is the ²¹²Bi radioactivity in a 250 μl aliquot identical to that used in the chromatography but left unseparated and counted as a standard. The intercept F⁰ at the separation time (t=0) is the fraction of ²¹²Bi that escapes the DOTA ligand upon β⁻ decay of [²¹²Pb(DOTA)]²⁻ (see, Mirzadeh et al., p. 6); and

FIG. 15 is a plot showing the post separation decay/growth of ²¹²Bi radioactivity in fractions obtained by Chelex-100 ion exchange chromatography of a 250 μl aliquot of a solution initially containing only Na₂[Pb(DOTA)] doped with ²¹²Pb. The NH₄I fraction contains DOTA-complexed metal ions; the EDTA fraction contains metal ions not complexed to DOTA and eluted by use of 0.1 M Na₂EDTA (see, Mirzadeh et al., p. 5).

### DETAILED DESCRIPTION OF THE INVENTION

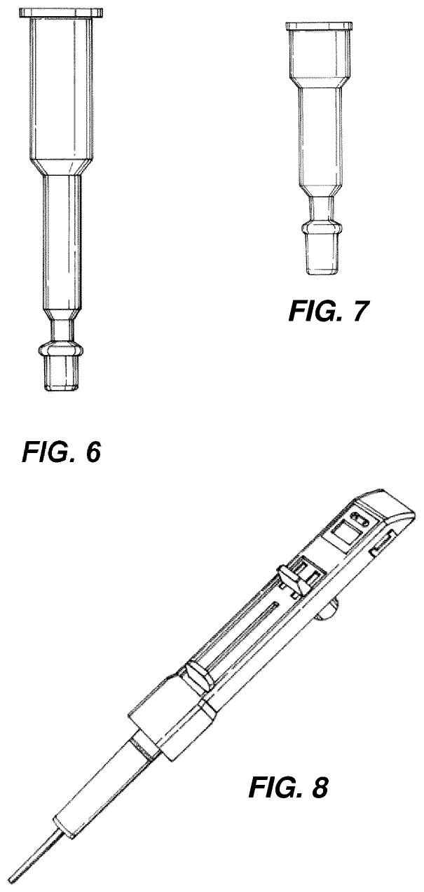

Reference will now be made to the drawings wherein like reference numerals identify similar structural features or aspects of the subject disclosure. For purposes of explanation and illustration, and not limitation, an illustrative view of an embodiment of a method and exemplary test assay in accordance with the disclosure is shown in FIGS. 6-13. Certain embodiments described herein can be used to reduce the likelihood of ejection of metals from a chelator during transition of radioisotopes, or said differently, embodiments can lower an energetic loss of a metal from the compound. Certain embodiments can be used to test chelators and chelator complexes for determining likelihood of metal ejection for improving the treatment of certain cancers, for example by reducing off-target dosing.

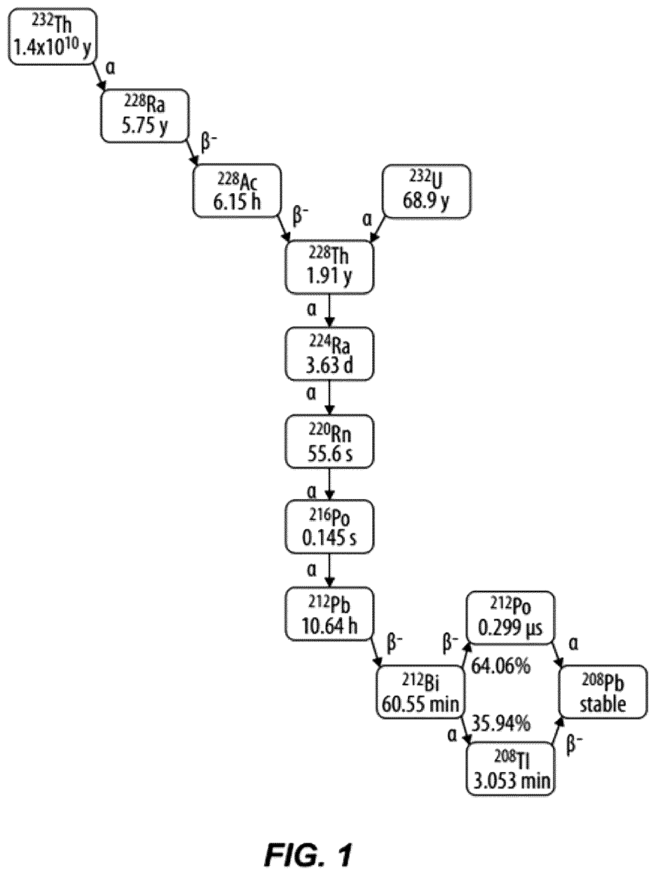

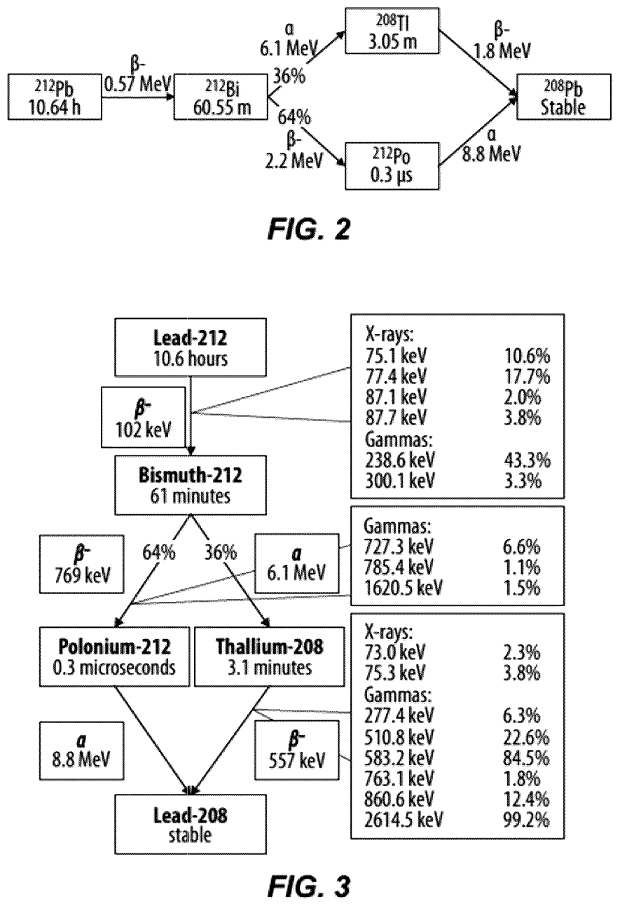

The use of Pb-212 in cancer treatment and diagnostics is becoming increasingly important because it simultaneously emits α and β particles, where the α particle emission are used to treat malignant cells and the R particle emissions permit SPECT/CT imaging. FIG. 1 shows the decay scheme from Th-232 to stable Pb-208 while FIG. 2 shows the decay scheme of Pb-212 to Pb-208, showing the relative energies at each decay step.

In addition to the α and β emissions, there are also copious photons released as shown in FIG. 3, with the highest energies coming from T1-208 (36% probability multiplied by the probabilities shown in FIG. 3). Alpha particle emission from a radioisotope is a powerful event, with energies typically in the range of 4-10 MeV, compared to 1-10 eV for individual chelator-metal binding energies. Thus, when an alpha emission occurs, there is essentially a 100% probability that the metal will be ejected.

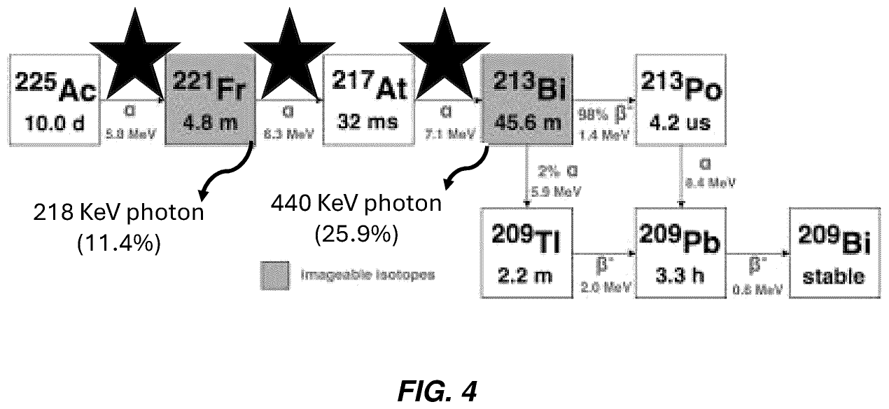

As an example, the decay of Ac-225 is shown in FIG. 4. Ac-225 has three a emissions prior to forming Bi-213 (as indicated by the stars in FIG. 4). Because of this, metal ejection is a serious concern with respect to treatments using Ac-225 as a radioisotope because as it decays, there will be a very low to zero chance that the Bi-213 will still chelated to its chelator, or associated with its targeting ligand-chelator complex, after that third a emission. This is problematic because the ejected Bi-213 can then be taken up in bone marrow and kidney rather than the targeted tumor cells and lead to high off-target radiation dose.

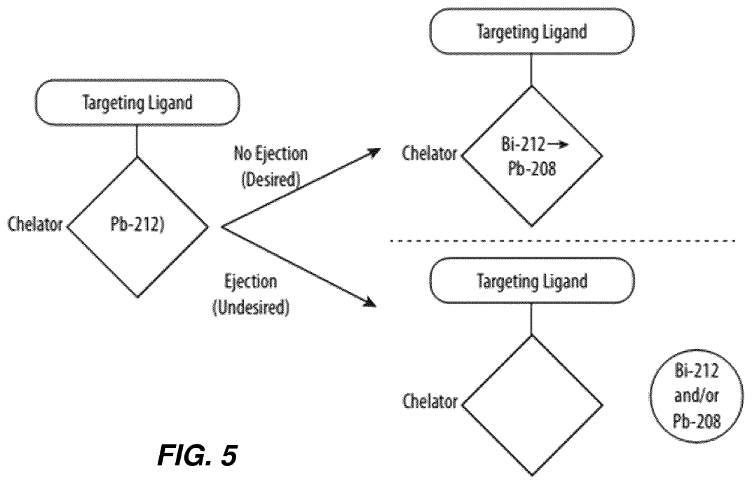

For Pb-212, the excess energy problem is more subtle. First, the alpha events (i.e., from Bi-212 to T1-208 or from Po-212 to Pb-208) occur after Bi-212 formation and are fast conversions (3 min and 0.3 ρs respectively). Therefore, these emissions are unlikely to have any impact on off-target uptake. It has been recently discovered that off-target dose from T1-208 show negligible ≤1% effects. However, the conversion of Pb2+-212 to Bi3+-212 has a 36.6% chance of going through a transient oxidation state as high or higher than 5+. This internal conversion (i.e., electronic conversion) is highly disruptive to the chelation of the metal to the chelator and is likely the cause of this “soft ejection” event. After ejection, the Bi-212 will no longer be associated with the chelator and therefore runs the risk of high bone marrow and kidney uptake, which is the problem the inventors strive to solve with the present invention. A schematic of this ejection event is shown in FIG. 5.

If the ejection event occurs after the drug (i.e., a targeting ligand plus chelator plus radioisotope) is already endocytosed inside the cell, then dissociation of the radioisotope from the chelator should have no biological effect. However, if the ejection event occurs in the bloodstream, before binding of the drug to the tumor even occurs, it could cause serious side effects due to uptake of the radioisotope in off-target tissues, such as bone marrow, lymph nodes, kidney, and elsewhere.

### Definitions and Additional Embodiments

The following definitions will be useful in understanding the instant invention.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used in the specification and claims, the singular form “a”, “an” and “the” include plural references unless the context clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish, parasites, microbes, and the like.

As used herein, the term “administration” or “administering” of the subject compound refers to providing a zwitterionic metal chelator of the invention and/or prodrugs thereof to a subject in need of diagnosis or treatment.

As used herein, the term “targeting vector” or “targeting ligand” refers to a moiety which is bound to or coordinated to the zwitterionic metal chelator of the invention to provide enhanced binding to particular cell types or an increased concentration in the presence of particular cell types. In certain embodiments, the targeting vector can be bound to the zwitterionic metal chelator in addition to the zwitterionic groups. In still other embodiments, the targeting vector can be bound to the zwitterionic metal chelator in place of one or more zwitterionic groups provided that the zwitterionic metal chelator retains at least one zwitterionic group.

As used herein, the term “therapeutic window” or “therapeutic index” refers to the relationship between the therapeutic and toxic dose of a given drug and is calculated using the ED50 and TD50 (Therapeutic Index=TD50/ED50). In certain embodiments of the invention, the zwitterionic metal chelators of the invention have a higher therapeutic index relative to other metal chelators. In certain other embodiments, the therapeutic window refers to a certainty safety factor (CSF) which is defined herein as the ratio of [TD₁/ED₉₉]. A CSF >1 indicates that the dose effective in 99% of the population is less than the dose that would be toxic in 1% of the population. In certain embodiments of the invention, the zwitterionic metal chelators of the invention have a higher CSF relative to other metal chelators.

As used herein, the term “carrier” refers to chemical compounds or agents that facilitate the incorporation of a compound described herein into cells or tissues.

As used herein, the term “acceptable” with respect to a formulation, composition or ingredient, as used herein, means having no persistent detrimental effect on the general health of the subject being treated.

As used herein, the term “diluent” refers to chemical compounds that are used to dilute a compound described herein prior to delivery. Diluents can also be used to stabilize compounds described herein.

As used herein, the term “zwitterionic group,” “zwitterionic ligand,” or “zwitterion” refer to one or more charged moieties or ligands which are present on or can be reacted with a metal chelator core. The zwitterionic metal chelators of the claimed invention are decorated by one or more zwitterionic groups, i.e., moieties combining positive (A+, FIG. 3) and negative charges (B−, FIG. 3) that sum to a net charge of zero. These zwitterionic groups are distinct from the chelator core itself, which typically has negative charges to chelate positively-charged metals. For example, a zwitterionic metal chelator with a chelator core of −4 that binds a +4 metal would have a total net charge of zero. Although a total net charge of zero is considered ideal, a zwitterionic metal chelator with a chelator core of −4 that binds a +2 metal, resulting in an overall charge of −2, would still be expected to exhibit improved properties in vivo because of shielding of the chelator core/metal complex by one or more zwitterionic groups. In the absence of zwitterionic groups, the molecule would have no such shielding or expanded water of hydration and would be more likely to bind non-specifically.

A particular active agent molecule may have several attached “zwitterionic groups” or charge pairs. In general, the anion portion and the cation portion of the zwitterionic group (charge pair) will be part of the same moiety, though it is possible for two ionic groups to be used as separate moieties to form a zwitterionic group. In particular embodiments, the zwitterionic group is covalently bound to the base structure via a carbon-carbon bond, a carbon-oxygen bond, or a nitrogen-carbon bond. Examples of zwitterionic groups (charge pairs) that can be included in the compounds and complexes of the claimed invention include, but are not limited to, ammoniophosphates, ammoniophosphonates, ammoniophosphinates, ammoniosulfonates, ammoniosulfates, ammoniocarboxylates, ammoniosulfonamides, ammonio-sulfon-imides, guanidiniocarboxylates, pyridiniocarboxylates, pyridiniosulfonates, ammonio(alkoxy)dicyanoethenolates, ammonioboronates, sulfoniocarboxylates, phophoniosulfonates, and phosphoniocarboxylates. The charged groups in these zwitterions can be separated by suitable spacer groups (C in FIG. 3) like linear or branched alkyl chains, aryl or heteroaryl moieties. In certain embodiments, the zwitterionic groups can be derivatives of amino acids, such as amino carboxylic acids, amino phosphonic acids, amino phosphinic acids or amino sulfonic acids, furthermore, aminoalkyl substituted sulfates or phosphates. Zwitterions can also be derivatives of betaines, such as carboxybetaines, sulfobetaines, sulfabetaines, phosphobetaines or phosphabetaines or N-oxides or derivatives of sulfamic acid. Particular examples of zwitterionic groups include ammonium sulfobetaines or N-oxides. A simple example of a zwitterionic group at physiological pH is the charge pair of a carboxylic acid (deprotonated at physiological pH) and an amine (protonated at physiological pH).

In some embodiments, the zwitterionic metal chelators of the invention can also comprise a targeting vector for an agricultural process, chemical process, disease, or tissue-specific epitope, such as the cyclic peptide cRGDyK (aka cRGD) bound to one or more arms of the metal chelator. cRGD is a cyclic derivative of the tripeptide Arg-Gly-Asp which can be conjugated to one or more arms of the metal chelators of the invention. In still other embodiments, the targeting vector is octreotide or bombesin. In other embodiments, the targeting vector is KUE or dPSMA-617, a small molecule capable of targeting Fibroblast Activation Protein (FAP) also called FAP-inhibitor or FAPI, an amino acid or combination of amino acids, or derivatives thereof. In such embodiments, the targeting vector-conjugates can be formed in place of one or more zwitterionic groups.

An ideal zwitterionic metal chelator conjugated to a targeting vector would adopt the total net charge of the targeting vector, which is purposeful because in most cases the charges on the targeting vector are crucial for the ability to bind its target. Targeted zwitterionic metal chelators thus retain the major advantage of minimizing non-specific binding while maximizing specific binding. It should be apparent to those skilled in the art that additional charges can be added to the zwitterionic metal chelator, if needed, to balance overall surface charge to zero.

In certain embodiments, the zwitterionic metal chelators of the invention chelator comprise a reactive linking group. Such reactive linking groups are typically an activated derivative of a carboxylic acid, such as an n-hydroxysuccinimide (NHS) ester, a sulfo-NHS ester, a pentafluorophenyl (PFP) ester, a hydroxybenzotriazole (HOBt) ester, a hydroxyazabenzotriazole (HOAt) ester, a tetrafluorophenyl (TFP) ester, an acid anhydride, an acid azide or an acid halide. Such reactive linking groups can be bound or substituted onto the chelator at any suitable structural location as would be understood by one of ordinary skill in the synthesis of such compounds. Reactive linking groups also include, but are not limited to, alkynes, azides, maleimides, thiols, amines, alcohols, phenols, carbonyls, phosphanes, alkenes and tetrazines.

As used herein, the term “contacting” refers to the bringing together of substances in physical contact such that the substances can interact with each other. For example, when an agent is “contacted” with tissue or cells, the tissue or cells can interact with the agent, for example, allowing the possibility of binding interactions between the agent and molecular components of the tissue or cells. “Contacting” is meant to include the administration of a substance such as an agent of the invention to an organism. Administration can be, for example, oral or parenteral.

As used herein, the term “ionic group” refers to a moiety comprising one or more charged substituents. The “charged substituent” is a functional group that is generally anionic or cationic when in substantially neutral aqueous conditions (e.g. a pH of about 6.5 to 8.0 or about physiological pH (7.4)). As recited above, examples of charged anionic substituents include anions of inorganic and organic acids such as sulfonate (—SO₃¹⁻), oxide, sulfinate, carboxylate, phosphinate, phosphonate, phosphate, and esters (such as alkyl esters) thereofin some embodiments, the charged substituent is sulfonate or oxide Examples of charged cationic substituents include quaternary ammonium ions (—NR₃⁺) and phosphonium ions (—PR₃⁺), where R is independently selected from C₁₋₆ linear alkyl, C₄₋₆ branched alkyl, C₃₋₆ cycloalkyl, aryl, heteroaryl and arylalkyl or heteroarylalkyl Other charged cationic substituents include protonated primary, secondary, and tertiary amines, as well as guanidinium or amidinium or pyridinium or other protonated, alkylated or oxygenated nitrogen heterocycles. In some embodiments, the charged substituent is —N(CH₃)₃⁺.

As used herein, the phrase “non-ionic oligomeric or polymeric solubilizing groups” refers to soluble polymers such as, for example, polyethylene glycol, polypropylene glycol, polyethylene oxide and propylene oxide copolymer, a carbohydrate, a dextran, polyacrylamide, a peptide and the like. The solubilizing group can be attached by any desired mode. The point of attachment can be, e.g., a carbon-carbon bond, a carbon-oxygen bond, or a nitrogen-carbon bond. The attachment group can be, e.g., an ester group, a carbonate group, an urea group, an alcohol group, an ether group, a sulfide group, an amino group, an alkylene group, an alkyne group, an azide group, a tetrazine, an amide group, a carbonyl group, or a phosphate group.

Some examples of solubilizing groups include polyethylene glycols, such as —(CH₂CH₂O)ₐ—H, —OC(═O)O(CH₂CH₂O)ₐH, —OC(═O)O(CH₂CH₂O)ₐCH₃, —O(CH₂CH₂O)ₐCH₃, and —S(CH₂CH₂O)₂CH₃, “a” being an integer between about 2 and about 250. In some embodiments, “a” is 4 to 12 or 5 to 10. In further embodiments, “a” is 6, 7, or 8. Other examples of solubilizing groups include dextrans such as —OC(═O)O(dextran).

The solubilizing moiety can have an absolute molecular weight of from about 500 amu to about 100,000 amu, e.g., from about 1,000 amu to about 50,000 amu or from about 1,500 to about 25,000 amu.

Further examples of solubilizing groups include: —(CH₂)_{c}—(OCH₂CH₂)_{d}—OR^{a}, wherein “c” is 0 to 6, “d” is 1 to 200, and R^{a} is H or C₁₋₆ alkyl. In some embodiments, “c” is 1 to 4, “d” is 1 to 10, and R^{a} is H. In some embodiments, “d” is 6 or 7.

See WO 2008/017074, U.S. Ser. No. 12/376,243 (filed Feb. 3, 2009), and U.S. Ser. No. 12/376,225 (filed Feb. 3, 2009), each of which is incorporated herein by reference in its entirety, for a further description of suitable non-ionic oligomeric or polymeric solubilizing groups, and method for incorporating them into dyes.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Compounds of the invention can also include all isotopes of atoms occurring in the intermediates or final compounds. Isotopes include those atoms having the same atomic number but different mass numbers. For example, isotopes of hydrogen include tritium and deuterium.

The chemical substances represented herein by name, chemical formula, or structure are meant to include all stereoisomers, geometric isomers, tautomers, resonance structures, and isotopes of the same, unless otherwise specified.

The chemical substances described herein may be charged or include substituents with formal charges. When such chemical substances are represented as charged, it is understood that, unless otherwise specified, the charges are generally countered with an appropriate counterion. For example, chemical substances or functional groups having a charge of −1 are understood to be countered with an ion have a +1 charge. Suitable counterions with +1 charge include Na+, K+, tetraalkylammonium ions, and the like. Conversely chemical substances or functional groups having a charge of +1 are understood to be countered with an ion having a −1 charge. Suitable counterions with −1 charge include F—, Cl—, Br—, I—, sulfate, phosphate, perchlorate, acetate, trifluoroacetate, maleate, fumarate, mesylate, lactate, pyruvate, levulinate, gluconate and the like.

### Zwitterionic Metal Chelators

In certain aspects, the disclosure is directed to the addition of zwitterionic groups to metal chelators coordinated to a chelating radiotherapeutics or radiodiagnostics for reducing off-target dosing by reducing the likelihood that the metal or metal isotope of the chelating radiotherapeutic or radiodiagnostic is de-chelated from the chelator complex prior to the chelating radiotherapeutic or radiodiagnostic reaching the target cells.

These zwitterionic metal chelators are useful in imaging, diagnostic, chemical processing, and treatment agents. Certain embodiments of zwitterionic metal chelators have desirable properties that maximize solubility in aqueous environments, minimize non-specific interactions, and retain the ability to target thus resulting in an improved performance in a variety of medical, agricultural, and chemical processes. In in vivo and medical applications, zwitterionic metal chelators improve the signal-to-background ratio and therapeutic window as compared to other metal chelators while retaining high stability.

Exemplary medical applications include applications for detection, imaging, or treatment in/of a subject or in a biological sample In certain embodiments, the disclosure further provides for treatment of benign and malignant tumors and tumor cells using a zwitterionic metal chelators labeled with a radioactive metal isotope known to emit ionizing radiation in a form that would result in the death of cells that take up the analogs labeled with the radioactive metal isotope.

In accordance with at least one aspect of this disclosure, a method comprises reducing a probability of de-chelation of a metal or metal isotope from a chelating radiotherapeutic, the chelating radiotherapeutic including a chelating radioisotope and a metal chelator, during one or more of: electronic conversion of the chelating radioisotope (i.e., change in oxidation state) and/or radioactive decay of the chelating radioisotope (i.e., alpha particle emission) by including one or more zwitterionic group on the metal chelator (e.g., one, two, three, or four, or more zwitterionic groups).

In certain embodiments, adding the one or more zwitterionic groups to the metal chelator, forms a zwitterionic metal chelator complex. In certain embodiments, the zwitterionic metal chelator complex includes the one or more zwitterionic groups, the metal chelator and the metal or metal isotope. In certain embodiments, the metal or metal isotope selected from the group consisting of a radionuclide, a label, a paramagnetic metal and a heavy metal.

In certain embodiments, the metal chelator can be or include one of (or a derivative of): a DOTA-based metal chelator, a DOTAM-based metal chelator, a PCT-based metal chelator, a DOTP-based metal chelator, a Amide-PCT-based metal chelator, a PCTP-based metal chelator, a PCTA-based metal chelator, a Gadopiclenol-based metal chelator, a deferoxamine-based metal chelator, a NOTA-based metal chelator, a PyC3A-based metal chelator, a macropa-based metal chelator, a PYTA-based metal chelator, or a porphyrin-based metal chelator. Specific structures of embodiments of metal chelators having one or more zwitterionic groups included thereon are shown and described next, however additional embodiments of metal chelators and detailed schemes for synthesizing one or more embodiments of the metal chelators disclosed herein are shown and described in U.S. patent application Ser. No. 18/640,169 filed Apr. 19, 2024 (now U.S. Pat. No. 12,440,585), Ser. No. 19/266,396 filed Jul. 11, 2025, Ser. No. 18/732,982 filed Jun. 4, 2024, PCT/US2024/045968 filed Sep. 10, 2024, Ser. No. 19/394,299 filed Nov. 19, 2025, 63/767,817 filed Mar. 6, 2025, and Ser. No. 19/558,087 filed Mar. 5, 2026, each of which are incorporated by reference herein in their entirety.

In certain embodiments, one or more zwitterionic groups can be added to a metal chelator or chelator complex based on DOTA or DOTAM. For example, in certain embodiments, the metal chelator can be based on 1,4,7,10-Tetraazacyclododecane-1,4,7,10-tetraacetic acid (DOTA), wherein the metal chelator can include the following:

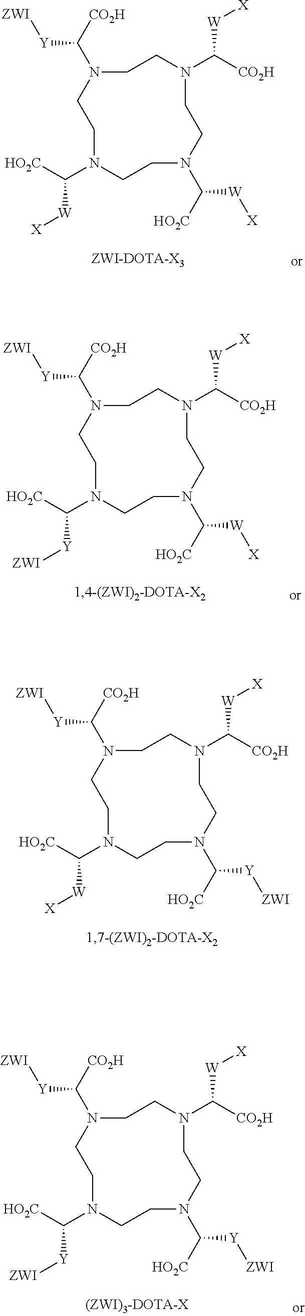

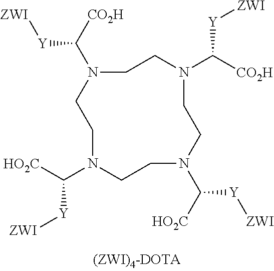

in which ZWI represents a zwitterionic group; each instance of W and Y independently represent a linking group, and each instance of X represents a reactive group. In certain embodiments, ZWI represents an ammonium sulfobetaine group or an N-oxide group optionally formed by click-chemistry (i.e. having a triazole ring).

In certain embodiments, one or more zwitterionic groups can be added to a metal chelator or chelator complex based on 1,4,7,10-Tetraazacyclododecane-1,4,7,10-tetraacetic amide (DOTAM), which is a derivative of DOTA in which —COOH groups are replaced with —CONR2 groups. Here, the metal chelator can be one of the following:

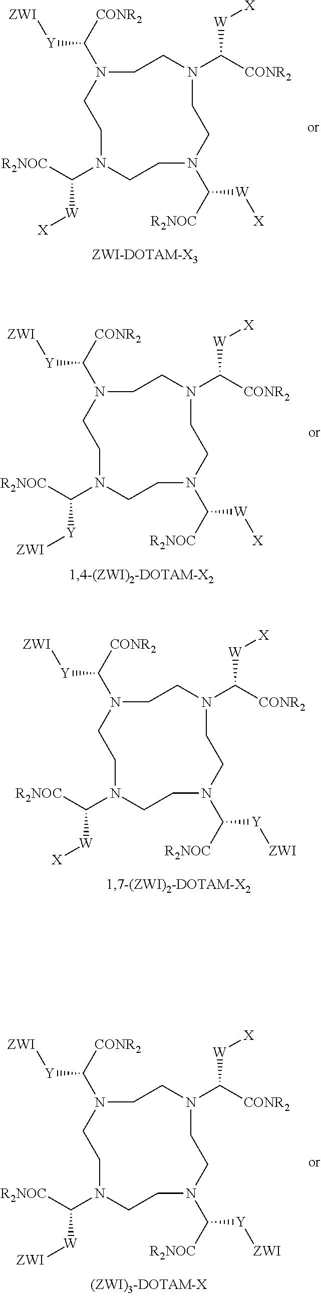

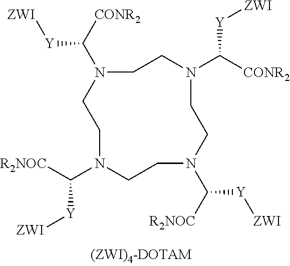

in which ZWI represents a zwitterionic group; each instance of W and Y independently represent a linking group, and each instance of X represents a reactive group. In certain embodiments, ZWI represents an ammonium sulfobetaine group or an N-oxide group optionally formed by click-chemistry (i.e. having a triazole ring). Substituents R are hydrogen or alkyl groups or a combination of both. In embodiments where the metal chelator is based on DOTA or DOTAM (or derivative thereof), the metal or metal isotope can be Pd, Pb, Zr, Cu, Ga, In, Y, Gd, Lu, Ac or Tb.

In certain embodiments, one or more zwitterionic groups can be added to a metal chelator or chelator complex based on 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;gadolinium(3+), also known as Gadopiclenol. In certain embodiments, one or more zwitterionic groups can be added to a metal chelator or chelator complex based on a derivative of 3,6,9,15-Tetraazabicyclo[9.3.1]pentadeca-1(15),11,13-triene-3,6,9-triacetic acid (PCTA). Accordingly, in certain such embodiments, the metal chelator can have the following structure:

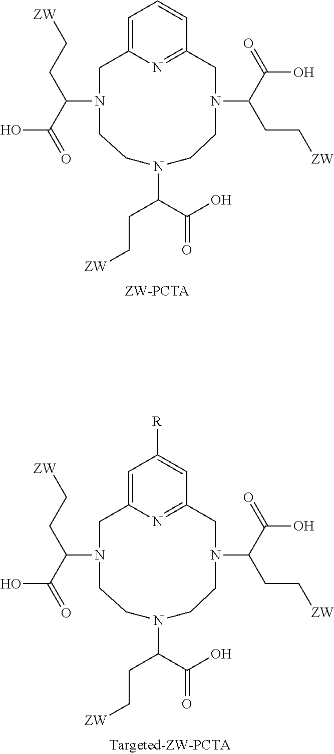

in which ZW represents a zwitterionic group. In certain embodiments, ZW represents an ammonium sulfobetaine group or an N-oxide group optionally formed by click-chemistry (i.e. having a triazole ring). In such embodiments the metal or metal isotope can be Gd or Gd³⁺, Pb, Mn, Zr, Cu, Ga, In, Y, Gd, Lu, c or Tb.

In certain embodiments, one or more zwitterionic groups can be added to a metal chelator or chelator complex based on Amide-PCTA, where Amide-PCTA is a derivative of PCTA in which —COOH groups are replaced with —CONR₂ groups. Such zwitterionic metal chelators include the following:

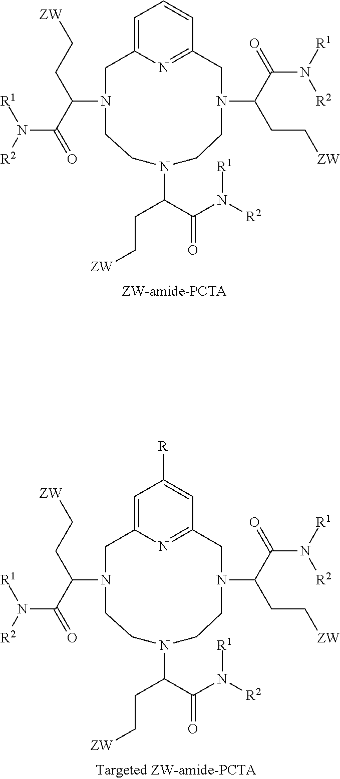

wherein ZW represents a zwitterionic group. Substituents R can be targeting vectors or reactive groups. Substituents R¹ and R² can be the same or different and are H, alkyl or aryl groups. In certain embodiments, substituents R¹ and R² are each H. In certain embodiments, ZW represents an ammonium sulfobetaine group or an N-oxide group optionally formed by click-chemistry (i.e. having a triazole ring) In such as where the metal chelator is based on amide-PCTA, the metal or metal isotope can be Gd, Gd³⁺, Pb, Mn, Zr, Cu, Ga, In, Y, Gd, Lu, c or Tb.

In certain embodiments, one or more zwitterionic groups can be added to a metal chelator or chelator complex based on 2-[3,9-bis[1-carboxylato-4-(2,3-dihydroxypropylamino)-4-oxobutyl]-3,6,9,15-tetrazabicyclo[9.3.1]pentadeca-1(15),11,13-trien-6-yl]-5-(2,3-dihydroxypropylamino)-5-oxopentanoate;gadolinium(3+), also known as gadopiclenol. In particular, the metal chelator is a derivative of 3,6,9,15-Tetraazabicyclo[9.3.1]pentadeca-1(15),11,13-triene-3,6,9-triacetic acid (PCTA), where —COOH groups are replaced with —PO(OH)2 groups, known as PCTP. Such zwitterionic metal chelators include the following:

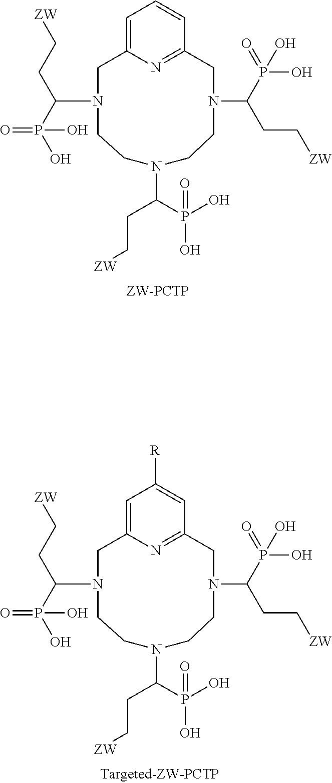

in which ZW represents a zwitterionic group. In certain embodiments, ZW represents an ammonium sulfobetaine group or an N-oxide group, optionally formed by click-chemistry (i.e. having a triazole ring). In such embodiments, the metal or metal isotope can be Pd, Pb, Mn, Zr, Cu, Ga, In, Y, Gd, Lu, Ac or Tb.

In certain embodiments, one or more zwitterionic groups can be added to a metal chelator or chelator complex based on 1,4,7,10-tetraazacyclododecane-1,4,7,10-(tetra(methylene phosphonic acid (DOTP), which is a derivative of DOTA in which —COOH groups are replaced with —PO(OH)2 groups. Such zwitterionic metal chelators include the following:

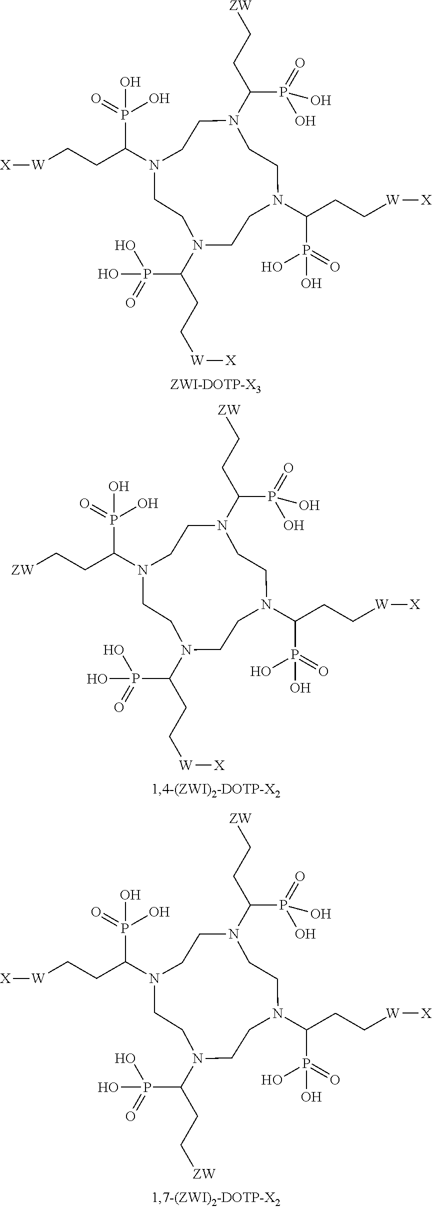

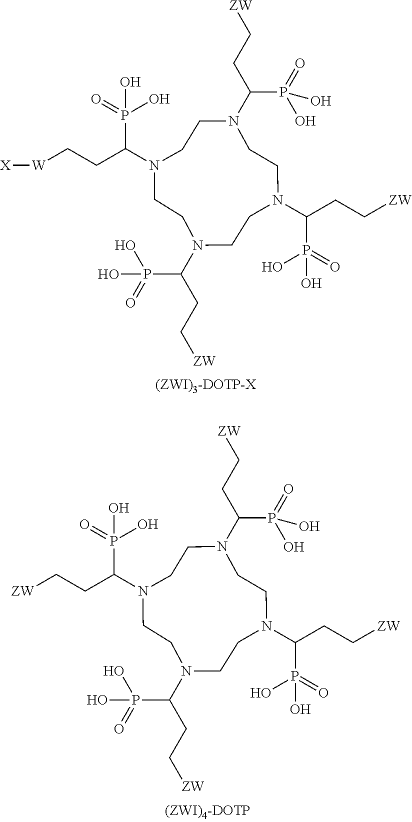

in which ZWI represents a zwitterionic group; each instance of W and Y independently represent a linking group, and each instance of X represents a reactive group. In certain embodiments, ZWI represents an ammonium sulfobetaine group or an N-oxide group, optionally formed by click-chemistry (i.e. having a triazole ring). Substituents R are hydrogen or alkyl groups or a combination of both. In such embodiments, the metal or metal isotope can be Pd, Pb, Zr, Cu, Ga, In, Y, Gd, Lu, Ac or Tb.

In certain embodiments, one or more zwitterionic groups can be added to a metal chelator or chelator complex based on N-picolyl-N,N′,N′-trans-1,2-cyclohexenediaminetriacetate (PyC3A), where the zwitterionic metal chelator is one of the following:

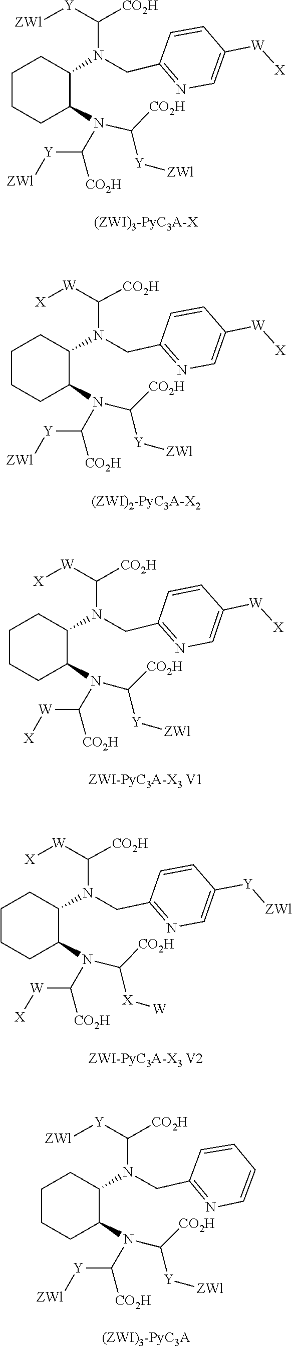

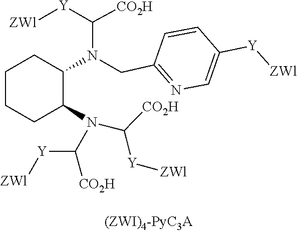

where the ZWI and the reactive groups X can be separated from the chelator core structure by appropriate spacer moieties W and Y including alkyl, aryl or heteroaryl groups. In certain embodiments, each of W and Y may independently be absent. In certain embodiments, ZWI represents an ammonium sulfobetaine group or an N-oxide group, optionally formed by click-chemistry (i.e. having a triazole ring). In such embodiments, the metal or metal isotope can be Mn.

In certain embodiments, one or more zwitterionic groups can be added to a metal chelator or chelator complex based on macropa, where the metal chelator can be one of the following:

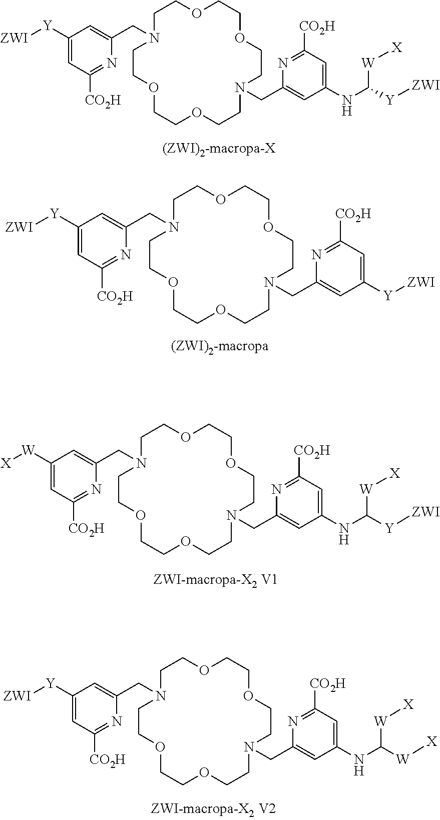

where zwitterionic groups ZWI and the reactive groups X can be separated from the chelator core structure by appropriate spacer moieties W and Y including alkyl, aryl or heteroaryl groups. In certain embodiments, each of W and Y may independently be absent. In certain embodiments, ZW represents an ammonium sulfobetaine group or an N-oxide group optionally formed by click-chemistry (i.e. having a triazole ring). In particular embodiments, the macropa-based zwitterionic metal chelator has the formula.

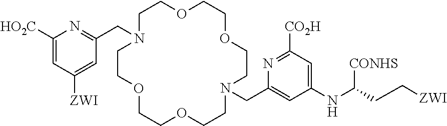

wherein ZWI represents a zwitterionic group. In certain embodiments, ZWI represents an ammonium sulfobetaine group or an N-oxide group, optionally formed by click-chemistry (i.e. having a triazole ring). In certain embodiments, such as where the metal chelator is based on macropa, the metal or metal isotope can be Ac 3+ or Bi 3+.

In certain embodiments, one or more zwitterionic groups can be added to a metal chelator or chelator complex based on NOTA, and can be one of the following:

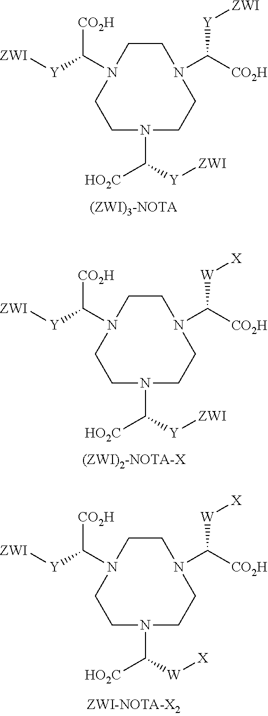

where zwitterionic groups ZWI and the reactive groups X can be separated from the chelator core structure by appropriate spacer moieties W and Y including alkyl, aryl or heteroaryl groups. In certain embodiments, each of W and Y may independently be absent. In certain embodiments, ZWI represents an ammonium sulfobetaine group or an N-oxide group, optionally formed by click-chemistry (i.e. having a triazole ring)

In particular embodiments, the NOTA-based metal chelator can have the formula:

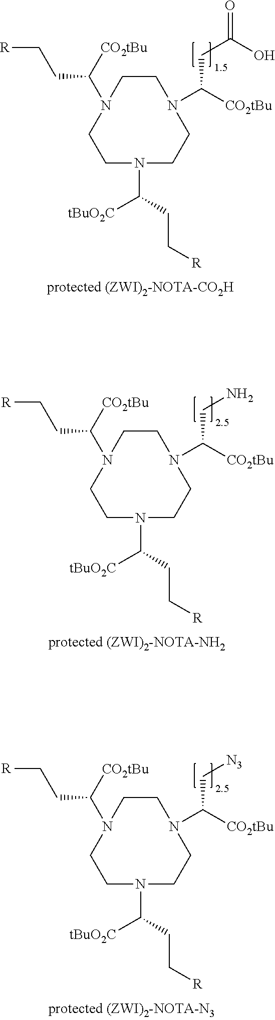

In certain embodiments, ZW represents an ammonium sulfobetaine group or an N-oxide group optionally formed by click-chemistry (i.e. having a triazole ring). In certain embodiments, such as where the metal chelator is based on NOTA, the metal or metal isotope can be Ga 3+, Cu 2+, Gd 3+, Ac 3+, or Bi 3+.

In certain embodiments, one or more zwitterionic groups can be added to a metal chelator or chelator complex based on deferoxamine, wherein the metal chelator is based on.

[chemical structure]

where ZWI and the reactive groups X can be separated from the chelator core structure by appropriate spacer moieties W and Y including alkyl, aryl or heteroaryl groups. In certain embodiments, each of W and Y may independently be absent. In still other embodiments, W-X can represent a hydrogen or an alkyl group. In certain embodiments, a deferoxamine-based zwitterionic metal chelator with four hydroxamate groups is derived from DFO* and one with three hydroxamate groups is derived from DFO. In certain embodiments, ZWI represents an ammonium sulfobetaine group or an N-oxide group, optionally formed by click-chemistry (i.e. having a triazole ring). In certain embodiments, such as where the metal chelator is based on deferoxamine, the metal or metal isotope can be Zr 4+, Fe 3+, Mn 2+, or Mn 3+.

In certain embodiments, one or more zwitterionic groups can be added to a metal chelator or chelator complex based on porphyrin, where the metal chelator is one of the following:

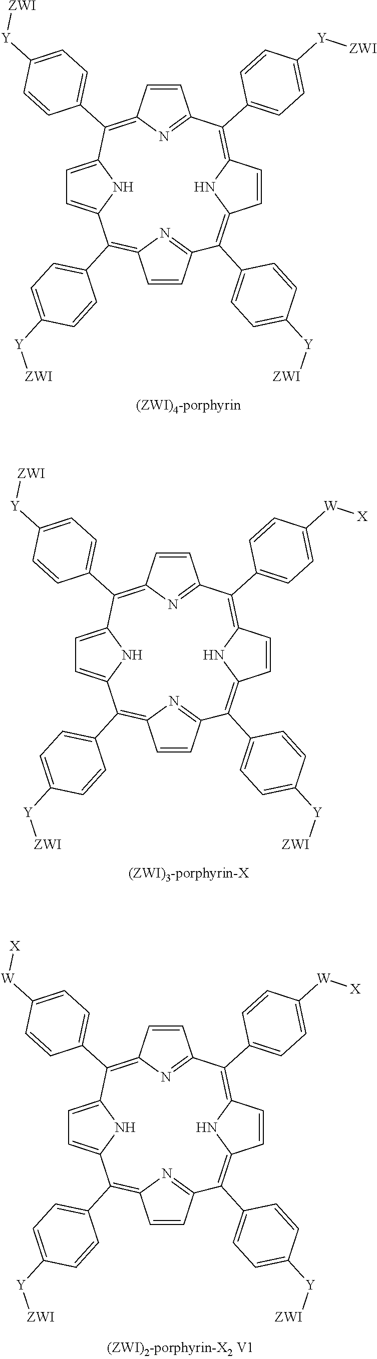

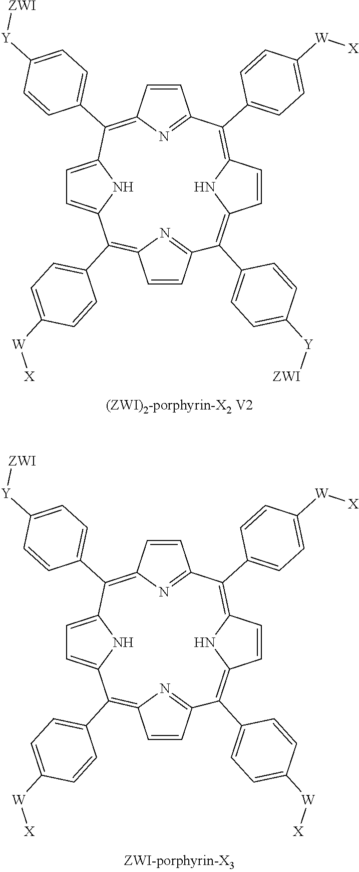

where zwitterionic groups ZWI and the reactive groups X can be separated from the chelator core structure by appropriate spacer moieties W and Y including alkyl, aryl or heteroaryl groups. In certain embodiments, each of W and Y may independently be absent. In particular embodiments, the porphyrin-based zwitterionic metal chelator includes 4 zwitterionic groups. In certain embodiments, ZWI represents an ammonium sulfobetaine group or an N-oxide group, optionally formed by click-chemistry (i.e. having a triazole ring).

In particular embodiments, the porphyrin-based metal chelator has the formula:

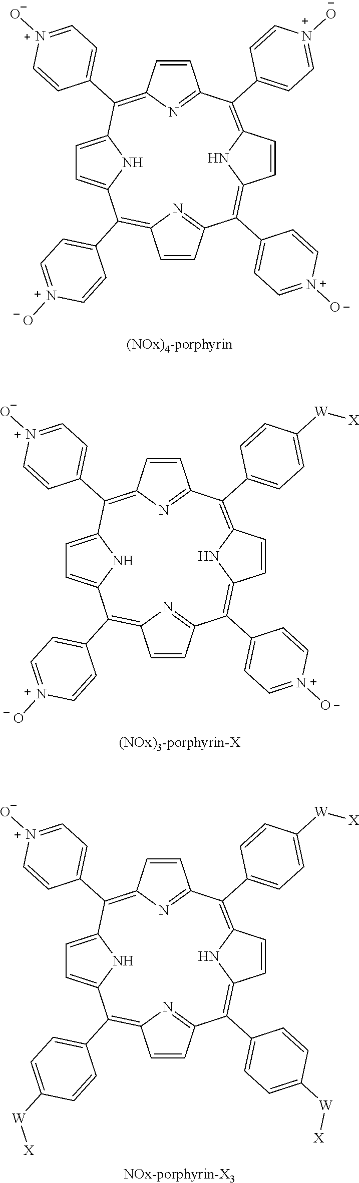

wherein the zwitterion is a pyridine-N-oxide (as drawn above) or an aromatic N-oxide derived from imidazole, pyrimidine or a similar heteroaromatic residue. X is a reactive group for conjugation to targeting vectors or other molecules or materials. In certain embodiments, such as where the metal chelator is based on porphyrin, the metal or metal isotope can be Mn 2+, Mn 3+, Fe 2+, Fe 3+, Gd 3+, Ac 3+, or Bi 3+.

In certain embodiments, the one or more zwitterionic groups can be added to a PYT metal chelator or chelator complex. For example, in certain embodiments, the PYT metal chelator or chelator complex can based on 3,6,10,13-tetraaza-1,8(2,6)-dipyridinacyclotetradecaphane-3,6,10,13-tetraacetic acid (PYTA) and can have one of the following structures:

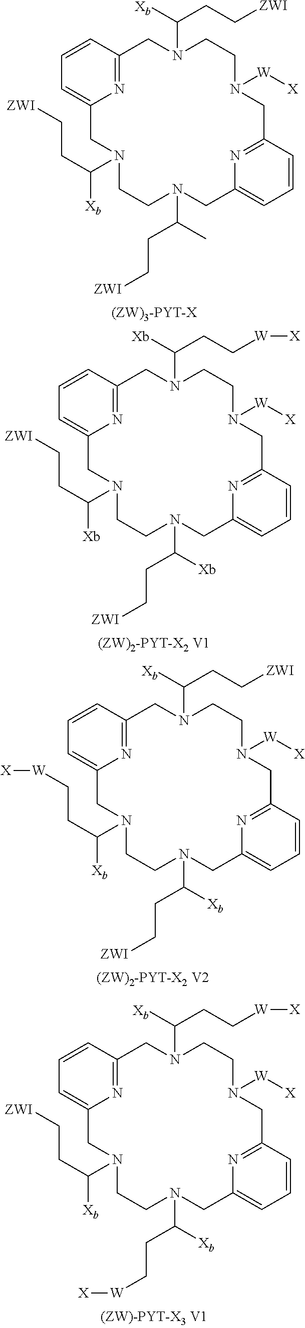

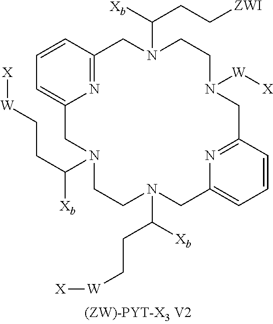

in which ZWI represents a zwitterionic group; each instance of W and Y independently represent a linking group, each instance of Xb may be —COOH or —PO(OH)2, and each instance of X represents a reactive group and/or a targeting vector. In certain embodiments, ZWI represents an ammonium sulfobetaine group, optionally formed by click-chemistry (i.e. having a triazole ring). In certain embodiments ZWI represents an N-oxide group, optionally formed by click-chemistry (i.e. having a triazole ring).

In certain embodiments, such as those in which each Xb is —COOH, the chelator may be referred to as a PYTA-based chelator. In certain embodiments, such as those in which each Xb is —PO(OH)2, the chelator may be referred to as a PYTP-based chelator. In certain such embodiments, such as where the one or more zwitterionic groups are added to a PYT based chelator, the metal or metal isotope can be Pb, Zr, Cu, Ga, In, Y, Gd, Lu, Ac, Tb Ac3+, Bi3+, or similar metal cations.

In certain embodiments, the chelating radiotherapeutic can further include one or more targeting vectors bound thereto. In certain embodiments, the one or more targeting vectors can be or include cRGD, KUE, PSMA-617, FAPI, octreotide, bombesin, or a homo- or hetero-dimer formed from their combination.

In certain embodiments, the method can further include, determining an energy delta between energy generated during transition of the chelated radioisotope and a binding energy of the metal or metal isotope to chelated radiotherapeutic and determining the probability of de-chelation of the metal or metal isotope during transition based on (or as a function of) a magnitude of the energy delta. The method can further include, including the metal chelator on the chelating radiotherapeutic and including one or more zwitterionic groups on the metal chelator, whereby inclusion of the one or more zwitterionic groups on the metal chelator reduces the probability of de-chelation of the metal or metal isotope during transition to zero or near zero. An exemplary embodiment of an inventive assay for testing certain metal chelators propensity to de-chelate is discussed next, in the “Metal Chelator Selection Experiments” section.

Accordingly, in view of this determination, including the metal chelator on the chelating radiotherapeutic and one or more zwitterionic groups on the metal chelator further comprises selecting the metal chelator as a function of its respective percentage of metal or metal isotope released from the respective chelator complex, for example.

Determining which the metal chelators have shown the lowest probability of de-chelation for the chosen base radioisotope can be used to then inform which metal chelator should be included on the chelating radioisotope. Including the one or more zwitterionic groups on the appropriately chosen metal chelator can therefore further increase or improve efficacy of the radiotherapeutic and/or reducing an off-target dose of the chelating radiotherapeutic. For example, selecting the proper metal chelator (e.g., that which has the lowest propensity for metal de-chelation) will decrease the probability that the metal or metal isotope will be de-chelated from the chelating radiotherapeutic in the patient's blood stream, before reaching a targeted tissue, meaning off-target radiation dose of the chelating radiotherapeutic is reduced or eliminated. Said differently, selecting the metal chelator having the lowest probability of metal or metal isotope de-chelation from the chelating radiotherapeutic increases the probability that the metal or metal isotope will remain bound to the chelating radiotherapeutic when the chelating radiotherapeutic reaches the target tissue thereby increasing efficacy of the chelating radiotherapeutic dose. Including the one or more zwitterionic groups on the metal chelator further enhances these advantageous effects.

### Metal Chelator Selection Experiments—Determining De-Chelation Probabilities

In the literature, Mirzadeh et al. quantified the “soft ejection” during the Pb-212→Bi-212 conversion when using DOTA as chelator. Subsequent papers have made passing reference to creating “more stable” chelators, such as DOTAM. But when DOTAM was studied directly, such as in Bauer et al., it was found that DOTAM had the same ejection ratio as DOTA published in Mirzadeh.

However, the assay used by Mirzadeh to test the ejection of bismuth when using the DOTA chelator is extremely cumbersome, requiring multiple micro-column purifications and exact timing. Accordingly, to overcome these challenges identified by the Mirzadeh assay, the inventors have developed a new and inventive test structure and semi-automated test method for testing new chelators for their resistance to recoil energy-based ejection of Bi-212. In certain embodiments, the test can use an R^{a}-224 generator as the source of Pb-212. In certain embodiments, the Pb-212 used in the assay can be isolated through the Rn-220 intermediate of a Th-228 generator. In certain embodiments, the Pb-212 can be isolated from a generator shown and described in U.S. patent application Ser. No. 19/542,002, filed Feb. 17, 2026, the entire content of which is incorporated by reference herein.

### Experimental Set Up, Materials, and Equipment

In Mirzadeh's test, 3×20 mm columns (0.14 mL bed volume) were used for separating complex from free ions, 2 mL (14 column volumes) of NH4I to flushed through Pb-DOTA complexes, washed with 1 mL of water (7 column volumes), then eluted free Pb from the Chelex using 2-3 mL (14-21 column volumes) of 100 mM Na2EDTA. Measuring the NH4I and Na2EDTA fractions provides the input needed to calculate free Bi-212, which was the purpose of Mirzadeh's experiment. However, Mirzadeh's experiment can be greatly improved and modernized, as will be discussed next with respect to the present invention.

In an exemplary embodiment of the an aspect of this invention, poly-prep columns having a maximum bed volume of 2 mL over a 3.9 cm working height (i.e., average column diameter of about 8.0 mm) are used for separating the complex from free ions. An exemplary embodiment of the poly-prep column is shown in FIG. 6. In this exemplary embodiment the poly prep column has an end cap at the proximal end, a reservoir, a working bed, a bed support, and a Luer fitting at the distal end. The reservoir can be a 10 mL volume reservoir, the working bed can be a 2 mL volume working bed, the bed support can be a porous 30 μm bed for retaining fine particles, and the Luer end fitting can include a snap off tip. In certain embodiments, such as shown, the height of the column (i.e., the reservoir plus the working bed) can be about 9 cm, where the reservoir is about 5 cm in height and the working bed is about 3.9 cm in height. To achieve 14 column volumes, a resin volume of 0.75 mL is used, which corresponds to a height of ≈1.5 cm. In certain embodiments, the resin volume can be chosen as a function of a maximum volume of a collection tube (e.g., in certain embodiments a Sarstedt tube). The resin volume can be chosen such that disruption of the resin when adding eluate is minimized or eliminated. In certain embodiments, 8 mL of eluant can be used, corresponding to 10.6 column volumes.

For each of the experiments which will be discussed further below, all well counter collection tubes will be filled to the same volume of 8 mL prior to counting.

In certain embodiments, a majority of the experiment involves pipetting multiple column volumes of solutions then collecting eluates at 9 evenly-spaced time points over 4 hours. To make this process as easy and fast as possible, which will also minimize exposure to radioactivity. Accordingly, embodiments of the invention can use a repeater pipette configured to automatically dispense the correct volume based on the tip inserted into the pipette and automatically count each dispensing step. An example of such a pipette is shown in FIG. 8, such as an Eppendorf M4 Repeater® available from Eppendorf, Inc, Hamburg Germany. In embodiments the pipette can be equipped with a 50 mL tip for most washing steps or a 10 mL tip for column preparation.

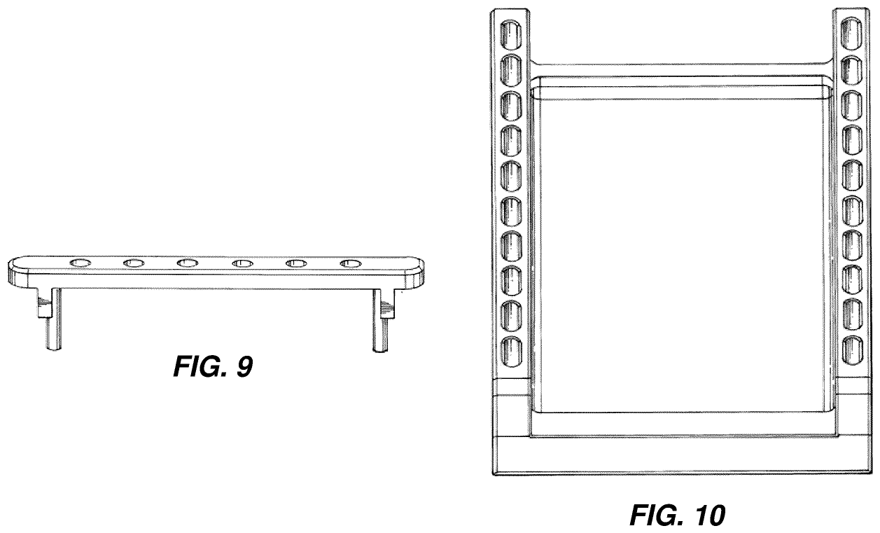

Referring now to FIG. 9, an exemplary embodiment of a column holder is shown. The column holders permit movement of columns from one type of rack to another, and from one collection tube to another, quickly and easily using either tongs or fingers. In a kit, a total of 10 column holders can be provided, one for preparing a reference solution and nine for individual time points. The column holder includes a body extending along an axis between a first end and a second end. A plurality of apertures are defined through the body in a direction perpendicular to the axis for accepting a plurality of prep columns, one per aperture. In certain embodiments, the apertures can be countersunk. The column holder further includes a first leg extending distally from a lower surface of the body at the first end, and a second leg extending distally from a lower surface of the body at the second end, where the legs extend in a direction perpendicular to the axis. The legs can be shaped for insertion into respective apertures in a column rack, which is described next with respect to FIG. 10. One or more extensions or tabs can be included, extending axially from the first end and the second end respectively for grabbing by a user when the column holder is placed in the column rack, for example.

With reference to FIG. 10, an exemplary embodiment of a column preparation rack is shown. The rack includes a lower body portion and two arms extending upward from the lower body portion. A plurality of bores are defined through an upper surface of each arm configured to accept the legs of the column holder. In the example shown in FIG. 10, 20 bores are defined, 10 on each arm, since 10 column holders are used. The column holders, when inserted, will extend across the lower body portion in a width direction and above the lower body portion. The lower body portion has a well or reservoir defined in a upper surface thereof for holding a liquid beneath the column holders, such as a chelex-100 slurry or 100 mM NH4I solution. The reservoir allows the user to quickly fill columns seated in the respective column holders using the fluid contained in the reservoir. Since the reservoir is disposed directly below the columns, and is a large open space, collecting and dispensing the liquids into the column can be made much easier and faster than if having to reload from a source container, for example. One or both of the arms can be indexed for labeling each column holder in the rack, for example, as shown, the first bores are labelled as the reference solution, and the remainder are labelled with their sample time (e.g., 0 min to 240 min, increasing at 30 min increments). In certain embodiments, a trough can be defined in the lower body portion to capture any fluid that flows through the columns. In certain embodiments, the trough can have a volume of greater than 400 mL, for example, about 456 mL. In certain embodiments, a fixing means can be included on an underside of the lower body portion configured to releasably fix the column rack to a work surface to prevent any movement or tipping of the rack while the columns are prepared. In certain embodiments, fixing means can include magnetic tape for example, which will be attracted to stainless steel benchtops (i.e., inside a hot cell).

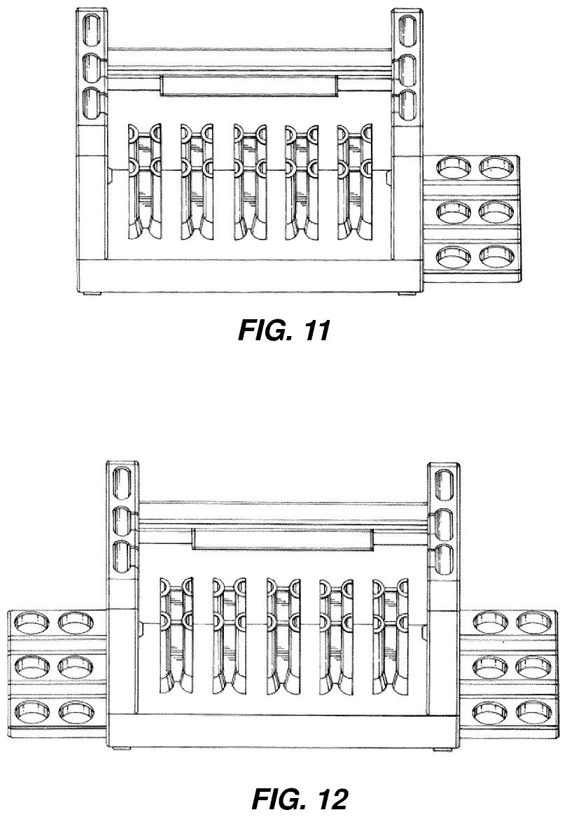

Shown in FIG. 11 is an exemplary embodiment of a reference solution rack which is configured to aiding in the preparation of the Pb-212 “Reference Solution” that is ultimately sampled every 30 minutes. The reference solution rack is similar to the column preparation rack except that it includes fewer slots for holding the column holders and additionally includes slots for holding reference tubes for each time point as well as their caps. As shown in FIG. 10, a rear position of the arm slots can be used for used columns, the middle position can be used for the reference solution, and the front position can be used for the current time point diluted reference. In certain embodiments, plurality of extensions can be included, extending upward from the upper face of the lower body portion and can be sized to hold individual collection columns for example. As shown in the example of FIG. 10, space for 12 columns is provided, six for the reference columns in the middle position and six for the time point diluted solutions in the front position. An extension can be included, extending laterally (in the width direction) from the lower body portion having recesses defined therein for holding the caps of the time point diluted samples.

Referring now to FIG. 12, an exemplary embodiment of a column separation rack is shown. The column separation rack permits the actual separation experiment to be performed easily at various time points The column separation rack can be similar to that of the reference solution rack, except that it can be indexed such that the rear position slots hold used columns, the middle position slots holds the complex collection columns, and the forward position holds the free ion collection columns. Here, two extensions can be included for holding the caps of the columns, for example one extension for holding the complex collection column caps, and a separate holder for the free ion collection column caps. In certain embodiments, each of the components shown in FIGS. 6-12 can be included in a single kit package, or a plurality of kit packages.

### Experimental Overview

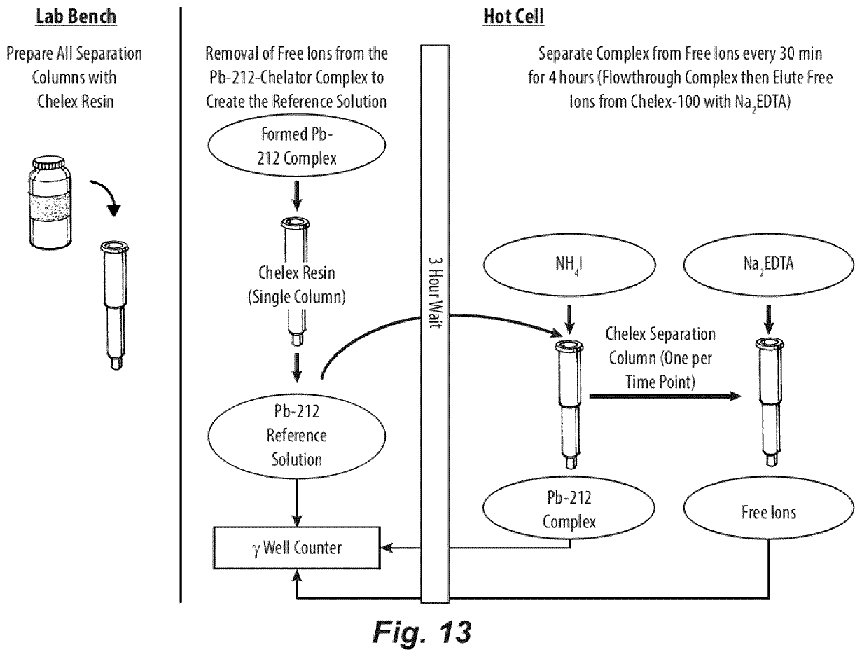

A schematic diagram of an exemplary embodiment of the experiment in accordance with one aspect of this disclosure is shown in FIG. 13 and is described further in this section. The left side of the diagram refers to work performed on the lab bench, i.e., outside the hot cell. There, all of the required reference solution columns (1 per chelator tested) and column separation columns (9 per chelator tested) are prepared by pipetting Chelex slurry into Poly-Prep columns using the column preparation rack and the semi-automated repeater pipettor. Inside the hot cell, on the right side of the diagram, the reference solution is prepared by removing any free Pb-212 using the reference solution rack. Two aliquots of this reference solution are used at each of the 9 time points. One aliquot (Ref) is measured directly in the well counter while the other is separated on a Chelex separation column and each fraction (flow-through and eluted) is counted separately. This process is repeated for each of the 9 time points.

There are two elements of timing that are critical to the success of this novel experiment. First, after preparation of the Pb-212 reference solution by passing it over a single Chelex column, the reference solution is held for 3 hours to ensure transient equilibrium of the Pb-212 (10.64 h half-life) and Bi-212 (1 h half-life). Second, after each separation column, eluates are held for exactly 10 min prior to well counting to permit transient equilibrium of the T1-208 daughter (3 min half-life), which provides the 583 keV photon that is measured by the well counter. Each step of the experiment will be discussed in detail in turn.

While detailed experimental protocol provided herein includes specific reference to particular radioisotopes, metal chelators, chelator complexes, reagents, and eluents, it should be appreciated by one having ordinary skill in the art having the benefit of this disclosure, that the experimental protocol can be modified as needed to accommodate testing other radioisotopes and other chelators or chelator complexes. The following description is intended for illustration and not limitation, and refers only to an exemplary embodiment of a protocol, using an exemplary metal chelator and radioisotope for the purpose of illustrating the protocol.

### Step 1—Preparation of Chelex-100 Resin Slurry

In embodiments, this step is performed the night before the experiment is conducted.

Swollen Chelex-100 resin will be needed in an amount of 10 time points*0.75 mL per chelator tested, i.e., 7.5 mL of packed resin for one chelator or 45 mL of packed resin for six chelators. Weigh out resin, place in large straight walled, wide mouth, graduated beaker, then add 5-volume excess (i.e., ≈45 mL if one chelator is being tested; 450 mL if six chelators are being tested) of 100 mM NH4I, pH 5-6 pre-pared using distilled H2O (dH2O). Mix well and let sit for 1 hour at room temperature. Pour off liquid, add an additional 5-volume excess of NH4I, and equilibrate at 4° overnight until beads are completely swollen. The next morning, pour off all liquid and add additional NH4I to create a 50% slurry. This 50% equilibrated slurry will be used for all experiments.

### Step 2—Preparation of All Columns

This step is performed outside the hot cell on any lab bench starting approximately 15-20 minutes prior to Reference Solution preparation (one Column Holder) and 15-20 minutes prior to Column Separation (remaining nine Column Holders).

Add magnetic stirrer to Chelex-100 50% slurry and stir vigorously to keep slurry well mixed. For each chelator being tested, place 1 column in each of the 10 black Column Holders. If only one chelator is being tested, place the column in the far left hole of each Column Holder. Label the front-most columns with chelator name and “Ref Sol”, e.g., “DOTA Ref Sol” or “ZCN-PCT Ref Sol”. These columns will be used to create the Reference Solutions. Label the other 9 rows of columns with the chelator and time point, for example “DOTA Time 0 min” in the first of 9 rows, “DOTA Time 30 min” in the second row and so on. Each position of the Column Preparation Rack is labeled on the left side.

Place a 10 mL disposable tip on the M4 repeater and cut off the end to permit easy pipetting of resin slurry. Aspirate 10 mL of 50% slurry then dispense 1.5 mL into each column. If there are 6 columns per Column Holder (i.e., if six chelators are being tested), dispense left to right (6×1.5 mL=9 mL per initial aspirant) within a Column Holder. If there is only one column per Column Holder, dispense front to back 5×1.5 mL=5 mL per initial aspirant). Dispense quickly so beads don't settle. Excess liquid will elute into the large reservoir leaving 0.75 mL of packed resin with a height of approximately 1.4 cm in-side the Poly-Prep column. Fill all columns needed.

### Step 3—Receipt, Storage, and Use of a R^{a}-224 Generator

If a Th-228 generator is being used, prepare Pb-212 as a 1 mCi/mL solution in 0.5 M NH4I and do not perform the remainder of step 3, move directly to step 4. If using an R^{a}-224 generator, the R^{a}-224 generator can only be stored and used in a hot cell with adequate negative pressure ventilation due to the release of radon gas upon whenever it is made dry.

In certain embodiments, the generator can be held with a standard ring clamp system in a vertical orientation with the inlet (e.g., a female Luer lock) facing up and the outlet (e.g., a male Luer lock) facing down. On the day the Ra-224 generator arrives, place it on ring stand in the hot cell and keep all Luer lock caps in place. In certain embodiments, the Ra-224 column has approximately 250 μL of resin in it, therefore 250 μL is equivalent to one column volume.

### Day 0 Cleaning Upon Receipt

Prepare two 1 cc syringes, one with 1 mL of 2 M HCl and one with 1 mL dH2O. Use the 2M HCl syringe to flush the inlet gently with the 1 mL of 2M HCl to expel generator contents into collection tube. The generator has now been washed free of stable Pb-208 and other buildup isotopes. Nex, flush with the 1 mL of dH2O to prepare the generator for storage overnight until the first milking on Day 1. Record the time and perform milking at same time each day.

Daily Preparation of Pb-212 from the Ra-224 Generator Prepare three 1 cc syringes, one with 0.5 mL of 2 M HCl, one with 0.25 mL dH2O, and one with 0.5 mL dH2O. Collect Pb-212 and replace solvent with NH4I using a Micro-Bio-Spin column (available from Bio-Rad Laboratories, Hercules CA), an exemplary embodiment of which is shown in FIG. 7.

Measure 1 g of Pb-resin beads, add to 15 cc tube, and swell with 10 mL of dH2O. Vortex. Decant super-natant and repeat. Snap the tip off of a Micro-Bio-Spin column then transfer beads to a height of 1 cm (400 μL of beads) above the frit. Centrifuge briefly in an ultrasmall microfuge (≥1,000 G) to remove all liquid. Place the spin column in a 2 cc (straight walled) Eppendorf tube. The spin column is now ready for the final Pb-212 purification.

Milk generator with 500 μL of 2M HCl followed by 250 μL of dH2O (750 μL total collected into the column). Agitate up and down several times to mix the Pb-212 with the beads. Let beads settle. While beads are settling, flush the outlet of the generator with 0.5 mL of dH2O to prepare the generator for overnight storage.

The Pb-212 in the spin column is now in the equivalent of 1.33 M HCl, which must be removed and re-placed with 0.1 M NH4I. Agitate up and down once again. Spin briefly to remove HCl and transfer spin column to a new 2 cc Eppendorf tube. Agitate beads again with 750 μL of dH2O. Spin to remove water then transfer spin column to a final 2 cc Eppendorf tube for final elution. Agitate beads with 500 μL of 1 M NH4I to ensure adequate contact with beads. Spin the column briefly to collect the final Pb-212 in NH4I into the Eppendorf collection tube. Confirm pH 5-6 using pH paper and 1 μL of eluant. Because of the construction of the Ra-224 generator, there is no need to perform AG-MP-50 resin cleanup of breakthrough Ra-224 (for example as required by Mirzadeh), since the resin is built into the bottom of the generator.

Assuming minimal loss during the purification, the expected yields of Pb-212 from a 2 mCi generator per day in 500 μL of final NH4I eluate is:

| | | | Volume | Volume |
|---|---|---|---|---|
| | | Concentration | Required | Required |
| | | (assuming | for 120 μCi | for 6 Chelators |
| | Expected | 0.5 mL | (One Chelator | Tested |
| Day | Yield | eluate) | Tested) | (720 μCi) |
| 1 | 1.41 mCi | 2.8 mCi/mL | 42.9 μL | 257 μL |
| 2 | 1.16 mCi | 2.3 mCi/mL | 52.2 μL | 313 μL |
| 3 | 0.96 mCi | 1.9 mCi/mL | 63.2 μL | 379 μL |
| 4 | 0.79 mCi | 1.6 mCi/mL | 75.0 μL | 450 μL |

Yield and activity concentration should be verified with a dose calibrator and volume required for 120 μCi adjusted accordingly. Specific activity, however, will be low, starting at only ≈0.13 MBq/nmol.

### Step 4—Preparation of the Pb-212-Chelator Complex

This preparation has a high concentration of cold Pb (low specific activity) and a 10% excess of Pb at the time of forming the Reference Solution. Using the excess of cold Pb to ensure there is control over the ratio of chelator to Pb creates very low specific activity complex, but does, in fact, ensure that the chelator is not in excess of the available Pb. This also eliminates the need to perform HPLC purification of the complex because passing the complex over a Chelex-100 column will remove any free Pb, which is otherwise in slight excess.

The stock solution of cold Pb-nitrate solution in the paper is 4.79 mM. It was diluted 37-fold and then 5-fold during mixture of the Pb-212 to a final concentration of 0.026 mM (26 μM) prior to adding DOTA such that total Pb was in a 10% excess. The table below shows exemplary stock solutions and dilutions for each chelator tested:

| | | | | | Final |
|---|---|---|---|---|---|
| | | | Concen- | | Concen- |
| | | | tration | Amount | tration |
| Order | | | Stock | to Form | Reagent |
| Added | Reagent | Stock | Solvent | Complex | (Solvent) |
| 1 | Radioactive | 1 | 0.5M | 120 μL | 120 μCi |
| | Pb-212 | mCi/mL | NH₄I | | (0.1M; |
| | (120 μCi) | | | | 60 μmol) |
| 3 | Cold Pb- | 1 mM | 100 | 15 μL | 0.025 mM |
| | nitrate | | mM HNO₃ | | (2.5 mM; |
| | | | | | 1.5 μmol) |
| 4 | Chelator | 1 mM | dH₂O | 13.5 μL | 0.0225 mM |

| | |
|---|---|
| Total Volume for Each Chelator Tested: | 451.5 μL |

Confirm pH 5 to 6 using pH paper. Adjust with HNO3 or NH₄I as needed

Repeat complex formation for each chelator to be tested. After preparing the mixture, heat at 85° C. for 15 min to form the complex then place on benchtop to cool to room temperature (≈10-15 min).

### Step 5—Preparation of the Reference Solution

For each condition being tested load one empty Sarstedt collection tube labeled “Ref Sol Chelator Name” (e.g., “Ref Sol DOTA” or “Ref Sol+ColdPb” or “Ref Sol HPLC”) into the middle row of the Reference Solution rack. Move the Ref Sol Column Holder with the desired number of columns inserted to the middle row of the Reference Solution rack, over the collection tubes. Transfer 0.5 mL of Pb-212-Chelator Complex from Step 4 to the column.

Using the repeater pipettor and a 50 mL tip, aspirate 50 mL of NH4I and elute columns with 7.5 mL (10 column volumes) of NH4I into the collection tube(s) underneath, which now contain 8 mL of the Reference Solution of pure Pb-212-Chelator complex without any free ions.

Lift up the Column Holder and touch the column tips to the inside of the collection tube to dispense any hanging drops then move the Column Holder to the rear position to get it out of the way. The front row of the Reference Solution rack should have no tubes in place as of yet. The middle row of the Reference Solution rack now contains the Reference Solution. Incubate for 3 hours in the hot cell before starting Separation Columns to ensure transient equilibrium between Pb-212 and Bi-212 (1 h half-life) is reached.

At the start of the 3-hour waiting period, transfer 31.25 μL (equivalent to 500 μL of the diluted separation samples in Step 6; 6.25 μCi) of the 100 μL of remaining complex from Step 4 to a collection tube containing 7.969 mL of NH4I (8 mL total) and count in the well counter programmed for Bi-212 to ensure that counting statistics for the experiment will be within range. Adjust count time as needed (shorter the better for actual experiment). Assume that counts will be up to 1.6-fold (7/10.6 half-lives of Pb-212) lower when the last time points are counted at 3+4=7 hours. If expected counting times are borderline, adjust activity levels for future experiments. These measurements also ensure that all of the reference solutions have the correct activity present.

### Step 6—Collection of Chelated Pb-212 and Free Ion Fractions Over Time

In this exemplary embodiment of the assay, the goal is to separate free Bi-212 from complexed Bi-212 over time and measuring the amount of each relative to a Reference Solution that is not separated on a column.

To begin, label three repeater pipette 50 mL tips “NH4I”, “dH2O”, and “EDTA”. They will be used throughout the experiment. Load the front row of the Reference Solution rack with one collection tube for every chelator being tested. Label tubes with chelator name, time point, and “Ref”, e.g., “DOTA 0 min Ref”. Add 7.5 mL of NH4I to each front row collection tube. Store caps on cap holder on the right side of the rack.

Load the middle row of the Column Separation Rack with one collection tube for each chelator being tested. This is the row to collect Pb-212 Chelator Complexes (i.e., flowthrough from the Chelex-100 res-in). Label tubes with chelator name, time point, and type, e.g., “DOTA 0 min Complex”.

Load the front row of the Column Separation Rack with one collection tube for each chelator being tested. This is the row to collect the Free Ions (i.e., Pb-212 that stuck to the column and will be eluted with Na2EDTA). Label tubes with chelator name, time point, and type, e.g., “DOTA 0 min Free Ions”.

Move the Column Holder for the current time point from the Column Preparation rack to the middle position (Complex Collection) of the Column Separation Rack.

To start the experiment transfer 0.5 mL from the respective Reference Solution in the middle row of the Reference Solution rack to the front row collection tube of the Reference Solution rack (Time Point Diluted Reference Solution), which should now contain a total of 8 mL of solution.

Transfer 0.5 mL from the same middle row of the Reference Solution rack (i.e., the Reference Solution) to the column sitting over the middle position of the Column Separation rack. Using the repeater pipettor and a 50 mL tip, aspirate 50 mL of NH4I and dispense 5 mL (6.6 column volumes) per column to result in 5.5 mL of eluate in each collection tube. Remove tip and save for next time point. Add new tip, aspirate 20 mL of dH2O and dispense 2.5 mL (3.33 column volumes) into the same columns, resulting in a total of 8 mL per complex collection tube. Remove tip and save for next time point.

Lift up the Column Holder and touch the column tips to the inside of the collection tube to dispense any hanging drops then move Column Holder to the front position (Free Ion Collection) of the Column Separation rack. Using the M4 pipettor and a 50 mL tip, aspirate 50 mL of 100 MM Na2EDTA and dispense 8 mL (10.3 column volumes) per column to result in 8 mL of eluate in each collection tube. Re-move tip and save for next time point.

Start timer and wait exactly 10 minutes before starting well counter. During wait, lift up the Column Holder and touch the column tips to the inside of the collection tube to dispense any hanging drops then move Column Holder to rear position of the Column Separation rack to get it out of the way. Cap all tubes and load well counter racks. This 10-minute delay permits Bi-212 to reach equilibrium with the T1-208 daughter (3.05 min half-life).

The well counter should be programmed to count both Bi-212 and Pb-212 using the following photons: Bi-212: 583 keV and Pb-212: 239 keV. The counting period should be as short as possible for good counting statistics, for example as optimized during Step 5.

Each chelator and time point will have 3 tubes to be counted: Reference (Ref), Complex, and Free Ion. Count all tubes in well counter after the 10 minute delay and prepare for next time point.

Caution should be taken not to program well counter for counting that takes too long because the T1-208 daughter is very short-lived (3.05 min) as is Bi-212 (60 min). Ideally the total count time should be roughly equivalent to the total time it took to perform all the column separations. Record counting period used for well counter: ______ sec/min. Repeat Step 6 every 30 minutes for a total of 4 hours (9 time points total).

### Step 7—Data Analysis

In this exemplary embodiment, the experiment will result in following time points and measurements as shown in the data collection table on the next page.

| | | | | | | Bi-212 | Corrected | Recovery = | | |
|---|---|---|---|---|---|---|---|---|---|---|
| | | | | | | from | Free | (A_{NH41} + | | |
| | | Reference | Complex | | Free Ion | Pb-212 | Ion; | A_{EDTA})/A_{2eq}; | | |
| | | Solution | Fraction | | Fraction | Break- | A₂ = | should | | |
| Time | Time of | (cpm); | (cpm); | f = | (cpm); | through | A_{EDTA} − | be = | F = | |
| Point | Separation | A_{2eq} | A_{NH4I} | A_{NH41}/A_{2eq} | A_{EDTA} | (A′) | A′ | 100% | A₂/A_{2eq} | In(F) |

| | |
|---|---|
| 1 | 0 |
| 2 | 30 min |
| 3 | 60 min |
| 4 | 90 min |
| 5 | 120 min |
| 6 | 150 min |
| 7 | 180 min |
| 8 | 210 min |
| 9 | 240 min |

| Average f: | _% | Avg Recovery: | _% |
|---|---|---|---|
| F0 = % Bi-212 Released = 1 − Average f: | % | | |

Note:
A′ is calculated by measuring Pb-212 in the Free Ion fraction and correcting for the amount of Bi-212 it would produce.

From the data collected in Table 3, calculate the average f, then F0=1−f, which will be the first estimate of the % Bi-212 released from the chelator. Mirzadeh reported 40% for F0 calculated this way.

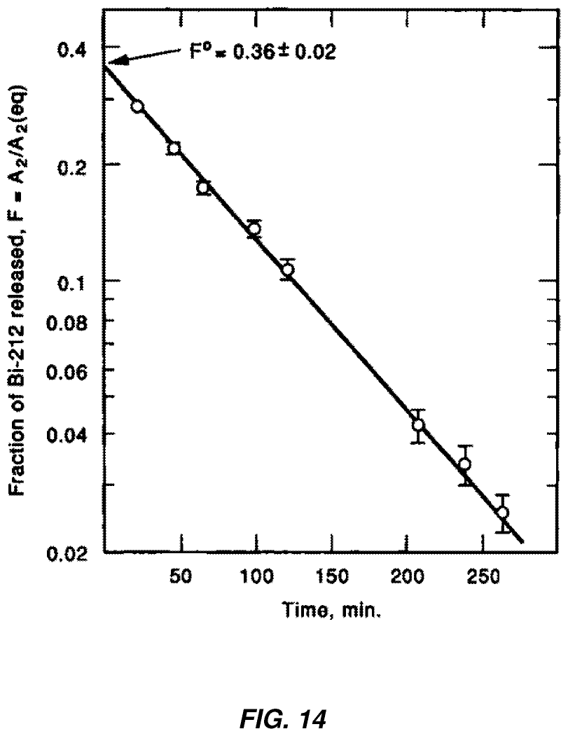

Separately, plot time in minutes on the X-axis and ln(F), that is ln(A2/A2eq) on the Y-axis. The Y-intercept is also F0, which is the fraction of released Bi-212 at time=0. When calculated this way, Mirzadeh reports F0=36%. An example plot, for example as found in Mirzadeh, is shown in FIG. 14.

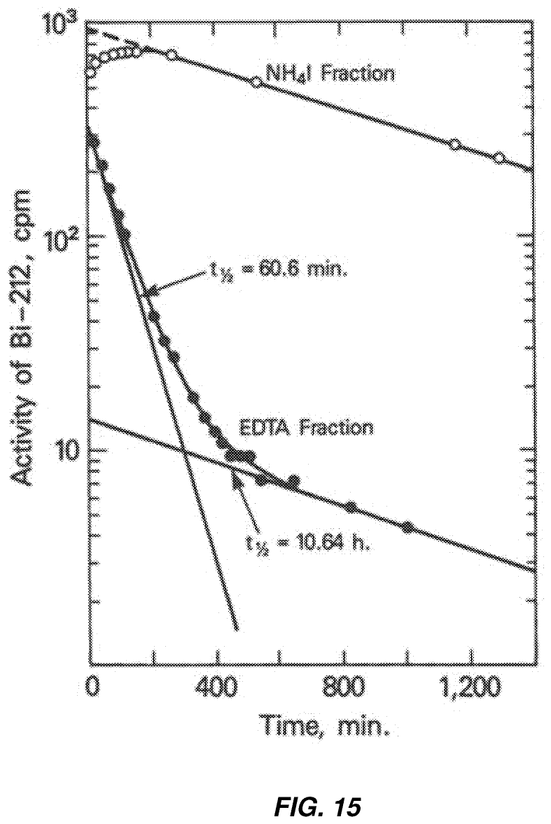

Taken together, Mirzadeh reported that 36-40% (range of the two different F0 measurements) of Bi-212 is ejected from DOTA due to recoil energy. Mirzadeh's entire dataset is visible on the plot shown in FIG. 15.

### Chelators to be Tested

Any one or more embodiments of the metal chelators disclosed herein can be tested with the inventive assay, including: DOTA, ZCN-DOTA (a zwitterionic version of DOTA), PCT (a DOTA derivative with three coordination sites that could have higher stability for Bi-212), ZCN-PCT (a zwitterionic version of PCT), DOTP, and/or ZCN-DOTP (a zwitterionic version of DOTP).

An exemplary test using DOTA can include, for example three columns per column holder, wherein: column 1 includes DOTA-Pb-212/DOTA-Pb-208 (1:1), column 2 includes DOTA-Pb-212/DOTA/Pb-208 (1:12.6), and column 3 includes DOTA-Pb-212 (HPLC purified).

With any chelator tested, a repeat of the best pair from Day 2 should be completed in triplicate. For example, if testing PCT and ZCN-PCT, and one or both showed lower recoil loss of Bi-212, then this pair would be tested again to confirm the results with 3 Reference tubes per chelator (6 total columns).

A fundamental flaw with the design of the original Mirzadeh experiment (which tested DOTA) is that chelator is present at a high concentration, thus if Bi-212 is released from DOTA during the 3 hour waiting period, there is a high probability of Bi-212 “re-finding” DOTA again prior to forming prior to column separation. This would result in an artificially low measured Bi-212 release in the assay, i.e., the assay suggests 36% loss but the true loss is much higher. Accordingly, the present disclosure and novel chelator test assay contemplates at least two ways to overcome this flaw in Mirzadeh's experiment by determining if re-formation is occurring.

First, excess cold Pb after formation of the Reference Solution could be added. The excess cold Pb would compete with any ejected Bi-212 for re-finding DOTA and thus increase the amount of Bi-212 as Free Ion.

Second, using HPLC Purified Complex. This condition can be created by purifying the DOTA-Pb-212 complex to high specific activity, creating roughly a >1,000-fold lower concentration of complex than in the original Protocol. Ejected Bi-212 as well as empty chelator will be at such a low concentration that the rate of the re-formation reaction would be much slower and the amount of Bi-212 as Free Ion would increase after separation.

If the original Protocol and new experiments (1) and (2) show the same values of F0 (≈36%) then that value is the true fractional loss from DOTA. If, however, (1) and (2) show a much higher F0 value then the fractional loss from DOTA is even higher and the 36% from the original paper was artificially low because of the conditions under which it was measured.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the apparatus and methods of the subject disclosure have been shown and described, those skilled in the art will readily appreciate that changes and/or modifications may be made thereto without departing from the scope of the subject disclosure.

1. A method, comprising:
reducing a probability of de-chelation of a metal or metal isotope from a chelating radiotherapeutic comprising a chelating radioisotope and a metal chelator during one or more of: electronic conversion of the chelating radioisotope and/or radioactive decay of the chelating radioisotope by including one or more zwitterionic groups on the metal chelator.

2. A method of improving efficacy and/or reducing an off-target dose of a chelating radiotherapeutic, comprising:
preparing a chelating radiotherapeutic having at least a chelating radioisotope and a metal chelator;
selecting the metal chelator for inclusion on the chelating radiotherapeutic based at least in part on a probability of de-chelation of a metal or metal isotope from the metal chelator during transition of the radioisotope; and
including one or more zwitterionic groups on the metal chelator.

3. The method of claim 2, wherein with the metal chelator is a DOTA-based chelator, a DOTAM-based chelator, a PCT-based chelator, a DOTP-based chelator, an Amide-PCT-based chelator, a PCTP-based chelator, a PCTA-based chelator, a Gadopiclenol-based chelator, a deferoxamine-based chelator, a NOTA-based chelator, a PyC3A-based chelator, a macropa-based chelator, a PYTA-based chelator, or a porphyrin-based chelator.

4. The method of claim 2, wherein the metal or metal isotope is one of: Pd, Pb, Zr, Cu, Ga, In, Y, Gd, Lu, Ac, Tb, Ga-66, Ga-67, Ga-68, Cu-64, Cu-67, Y-86, Co-55, Zr-89, Sr-83, Mn-52, As-72, Sc-44, Gd-153, Co-57, In-111, Ac-225, Tc-99m, Tc-94m, Re-186, Re-188, Y-90, Bi-213, Sm-153, Ho-166, Lu-177, Sc-43, Sc-44, Sc-47, Tb-149, Tb-152, Tb-155, Tb-161, Dy-166 or Pb-212, Gd or Gd³⁺, Ac 3+ or Bi 3+, Ga 3+, Cu 2+, Zr 4+, Fe 3+, Mn 2+, or Mn 3+.

5. The method of claim 2, wherein with the one or more zwitterionic groups included on the metal chelator are selected from the group consisting of: ammoniophosphates, ammoniophosphonates, ammoniophosphinates, ammoniosulfonates, ammoniosulfates, ammoniocarboxylates, ammoniosulfonamides, ammonio-sulfon-imides, guanidiniocarboxylates, pyridiniocarboxylates, pyridiniosulfonates, ammonio(alkoxy)dicyanoethenolates, ammonioboronates, sulfoniocarboxylates, phophoniosulfonates, phosphoniocarboxylates, amino carboxylic acids, amino phosphonic acids, amino phosphinic acids, amino sulfonic acids, aminoalkyl substituted sulfates, aminoalkyl substituted phosphates, betaines, N-oxide, or derivatives of sulfamic acid, and combinations thereof.

6. The method of claim 5, wherein the one or more zwitterionic groups include sulfobetaines or N-oxides.

7. The method of claim 6, wherein the one or more zwitterionic groups include:
an ammonium sulfobetaine group or an ammonium sulfobetaine group bound to the metal chelator through a triazole ring, or
an N-oxide group or an N-oxide group bound to the metal chelator through a triazole ring.

8. The method of claim 4, wherein metal chelator is a DOTA-based metal chelator, or a DOTAM-based metal chelator and the metal or metal isotope is Pd, Pb, Zr, Cu, Ga, In, Y, Gd, Lu, Ac or Tb.

9. The method of claim 4, wherein the metal chelator is a PCTA-based metal chelator and the metal or metal isotope is Gd or Gd³⁺, Pb, Mn, Zr, Cu, Ga, In, Y, Gd, Lu, c or Tb.

10. The method of claim 4, wherein the metal chelator is an amide-PCTA-based metal chelator and the metal or metal isotope is Gd, Gd³⁺, Pb, Mn, Zr, Cu, Ga, In, Y, Gd, Lu, c or Tb.

11. The method of claim 4, wherein the metal chelator is a PCTP-based metal chelator and the metal or metal isotope is Pd, Pb, Mn, Zr, Cu, Ga, In, Y, Gd, Lu, Ac or Tb.

12. The method of claim 4, wherein the metal chelator is a DOTP-based metal chelator and the metal or metal isotope is Pd, Pb, Zr, Cu, Ga, In, Y, Gd, Lu, Ac or Tb.

13-17. (canceled)

18. The method of claim 4, wherein the metal chelator is a PYT-based metal chelator, including a PYTA-based metal chelator or a PYTP-based metal chelator, and the metal or metal isotope is Pb, Zr, Cu, Ga, In, Y, Gd, Lu, Ac, Tb Ac3+, Bi3+.

19. The method of claim 2, wherein the chelating radiotherapeutic further comprises one or more targeting vectors.

20. The method of claim 18, wherein the one or more targeting vectors include cRGD, KUE, PSMA-617, FAPI, octreotide, bombesin, or a homo- or hetero-dimer formed from their combination.

21. The method of claim 2, wherein transition of the radioisotope includes one or more of: electronic conversion of the radioisotope or alpha particle emission from the radioisotope during radioactive decay of the radioisotope, the method further comprising:
determining an energy delta between energy generated during transition of the radioisotope and a binding energy of the metal or metal isotope to the metal chelator;
determining the probability of de-chelation of the metal or metal isotope during transition based on a magnitude of the energy delta; and
including the zwitterionic group on the metal chelator whereby inclusion of the zwitterionic group reduces the probability of de-chelation of the metal or metal isotope during transition to zero or near zero.

22. The method of claim 21, wherein determining the probability of de-chelation of the metal or metal isotope during transition based on a magnitude of the energy delta further comprises:
preparing a reference solution having a radioisotope-chelator complex, wherein the chelator complex is a reference chelator complex, measuring a radioactivity of the reference solution over a count period, and repeating the measuring over a plurality of time sampling intervals;
preparing a test solution having a radioisotope-chelator complex, wherein the chelator complex is a test chelator complex, measuring a radioactivity of the test solution over the count period, and repeating the measuring over the plurality of time sampling intervals;
calculating an average radioactivity for each of the reference solution and the test solution over the plurality of sampling intervals; and
calculating a percentage of metal or metal isotope released from the test chelator complex by dividing the average radioactivity of the test solution by the average radioactivity of the reference solution for each sampling interval.

23. The method of claim 22, further comprising, preparing a plurality of test solutions, each test solution having a radioisotope-chelator complex, wherein the radioisotope-chelator complex is a respective test chelator complex, and wherein each respective test chelator complex is different from one another, measuring a radioactivity of each test solution over the count period, and repeating the measuring over the plurality of time sampling intervals for each test solution.

24. The method of claim 23, further comprising, comparing the percentage of metal or metal isotope released from the chelator complex for each respective test solution to the reference solution to determine which respective chelator complex has a lowest percentage of metal or metal isotope released from the respective chelator complex.

25. The method of claim 24, wherein selecting the metal chelator for inclusion on the chelating radiotherapeutic includes selecting the metal chelator as a function of its respective percentage of metal or metal isotope released from the respective chelator complex.

26. The method of claim 2, wherein selecting the metal chelator for inclusion on the chelating radiotherapeutic based at least in part on a probability of de-chelation of a metal or metal isotope from the metal chelator during transition of the radioisotope further includes selecting a metal chelator having a lowest probability of de-chelation of the metal or metal isotope from the chelating radiotherapeutic.

27. The method of claim 26, further comprising:
administering the chelating radiotherapeutic to a patient intravenously; and
wherein selecting the metal chelator having the lowest probability of de-chelation of the metal or metal isotope from the chelating radiotherapeutic during transition of the chelating radioisotope decreases the probability that the metal or metal isotope is de-chelated from the chelating radiotherapeutic in the patient's blood stream, before reaching a targeted tissue thereby reducing an off-target radiation dose of the chelating radiotherapeutic.

28. The method of claim 27, wherein selecting the metal chelator having the lowest probability of de-chelation of the metal or metal isotope from the chelating radiotherapeutic increases the probability that the metal or metal isotope will remain bound to the chelating radiotherapeutic when the chelating radiotherapeutic reaches the target tissue thereby increasing efficacy of the chelating radiotherapeutic dose.

29. An improved radiopharmaceutical for the treatment and/or diagnosis of cancer in a patient, comprising;
a chelating radiopharmaceutical having a chelating radioisotope;
a metal chelator included on the chelating radioisotope and one or more zwitterionic groups included in the metal chelator, the metal chelator selected on a basis of a probability of de-chelation of a metal or metal isotope from the chelating radiopharmaceutical during one or more of: electronic conversion of the chelating radioisotope and/or due to excess energy generated from alpha particle emission from radioactive decay of the chelating radioisotope, where inclusion of the one or more zwitterionic groups reduces the probability of de-chelation, thereby decreasing an off-target radiation dose and increasing targeted cell uptake of the dose and maximizing an efficacy of the radiopharmaceutical.

30-32. (canceled)

33. The method of claim 29, wherein the radiopharmaceutical includes a radiodiagnostic being both an alpha- and beta-emitter and adapted for dosimetry by single-photon emission computed tomography (SPECT) imaging, and wherein inclusion of the one or more zwitterionic groups on the metal chelator reduces the probability of de-chelation of the metal or metal isotope from the radiodiagnostic after beta emission thereby increasing accuracy of dosimetry.

34-36. (canceled)
